(19) United States
(12) Patent Application Publication
(10) Pub. No.: US 2023/0160897 A1
(43) Pub. Date: May 25, 2023

(54) METHOD AND SYSTEM FOR IDENTIFYING COLON CANCER-SPECIFIC VESICLE-ASSOCIATED PROTEINS FROM TISSUE SAMPLE

(71) Applicants: Jan LOTVALL, GOTHENBURG (SE); Aleksander CVJETKOVIC, GOTHENBURG (SE); Cecilia LASSER, GOTHENBURG (SE)

(72) Inventors: Jan LOTVALL, GOTHENBURG (SE); Aleksander CVJETKOVIC, GOTHENBURG (SE); Cecilia LASSER, GOTHENBURG (SE)

(21) Appl. No.: 17/995,325

(22) PCT Filed: Mar. 31, 2021
(86) PCT No.: PCT/IB2021/052688

§ 371 Date: Oct. 3, 2022

Related U.S. Application Data
(60) Provisional application No. 63/004,696, filed on Apr. 3, 2020.

(51) Int. Cl. G01N 33/574; G01N 33/68

(52) U.S. Cl. CPC G01N 33/57419; G01N 33/6848; G01N 33/6842; G01N 2560/00

(57) ABSTRACT

There is disclosed a method of identifying colon cancer-specific vesicle-associated proteins from a tissue sample of a subject. The method comprises isolating tissue-resident extracellular vesicles from the tissue sample; identifying vesicle-associated proteins associated with the isolated tissue-resident extracellular vesicles; quantifying the identified vesicle-associated proteins to identify one or more major vesicle-associated proteins; creating vesicle-associated protein profiles for the identified vesicle-associated proteins; and comparing the vesicle-associated protein profiles for the identified vesicle-associated proteins with pre-determined vesicle-associated protein profiles of the tumour tissue samples and/or non-tumour tissue samples.

### TECHNICAL FIELD

The present disclosure relates generally to identification of biomarkers associated with diseases, and more specifically to identification of colon-cancer-specific vesicle-associated proteins. In particular, the present disclosure relates to methods and systems for identifying colon cancer-specific vesicle associated proteins from tissue samples of subjects. Furthermore, the present disclosure also relates to computer program products comprising non-transitory computer-readable storage medium having computer-readable instructions stored thereon, the computer-readable instructions being executable by a computerized device comprising processing hardware to execute the aforementioned methods.

### BACKGROUND

Colon cancer is one of the most commonly occurring cancers amongst both women and men (wcrf.org, January 2019). The year 2018 alone saw over a million new cases of colorectal cancer and over half a million deaths as a consequence of it. Therefore, there is a significant need for biomarker identification and for a deeper understanding of the mechanisms of colon cancer. Typically, EVs offer higher insight into cellular communications that occur in tumour microenvironments. Recent advances in cancer diagnosis and prognosis have employed extracellular vesicles (EVs) as potential biomarkers.

Conventionally, EVs have been chosen as diagnostic biomarkers for detecting specific diseases, including cancer. However, one or more types of protein associated with the EVs may be expressed by many types of cells and thus may not be associated with a specific disease. Moreover, isolation of disease-specific vesicle-associated proteins is difficult and isolated (namely, purified) vesicle-associated proteins may not be substantially pure. Therefore, membrane protein characteristics, such as intensity and quantity of vesicle-associated proteins, may not be accurate. Furthermore, the vesicle-associated protein profiles created using such isolated vesicle-associated proteins may not yield accurate or desired outcomes or results. In addition thereto, the conventional methods are complex and time-intensive.

Therefore, in light of the foregoing discussion, there exists a need to overcome the aforementioned drawbacks associated with conventional profiling of vesicle-associated proteins.

### SUMMARY

The present disclosure seeks to provide an improved method for identifying colon cancer-specific vesicle-associated proteins from a tissue sample of a subject and use thereof as biomarkers associated with colon cancer.

According to a first aspect, the invention is realized by a method of identifying colon cancer-specific vesicle-associated proteins from a tissue sample of a subject, characterized in that the method comprises the steps of:
(a) isolating tissue-resident extracellular vesicles from the tissue sample;
(b) identifying vesicle-associated proteins associated with the isolated tissue-resident extracellular vesicles;
(c) quantifying the identified vesicle-associated proteins to identify one or more major vesicle-associated proteins;
(d) creating vesicle-associated protein profiles for the identified vesicle-associated proteins; and
(e) comparing the vesicle-associated protein profiles for the identified vesicle-associated proteins with pre-determined vesicle-associated protein profiles of the tumour tissue samples and/or non-tumour tissue samples.

The present invention is of advantage in that it overcomes the highlighted drawbacks and provides a new methodology for identifying colon-cancer-specific vesicle-associated proteins from a tissue sample of a subject. The identified vesicle-associated proteins are sensitive as biomarkers for colon cancer. Moreover, the identified vesicle-associated proteins overcome the problem of irrelevant readouts associated with the conventional disease diagnosis in general and biomarkers in particular, and are distinguished from confounding factors through rigorous isolation, purification and identification schemes for use. In addition, the identified vesicle-associated proteins are isolated directly from an affected tissue and therefore mimic the cellular vesicle secretome at their highest concentration, and thus are potentially more likely to facilitate a detectable and reliable signal for biomarker identification. Furthermore, the vesicle-associated proteins as per the present invention have been identified specifically for colon cancer detection, and were found to be specifically up-regulated in tumour tissue samples as compared to the normal mucosal (or non-tumour) tissue samples. Additionally, the identified vesicle-associated proteins as per the present invention are convenient to isolate and are of high quality for use as sensitive diagnostic biomarkers.

Optionally, at the step (a) of isolating tissue-resident extracellular vesicles, the method includes: (i) processing the tissue sample to obtain a tissue conditioned fluid; (ii) collecting the tissue-resident extracellular vesicles from the tissue conditioned fluid; and (iii) purifying the collected tissue-resident extracellular vesicles by a density gradient preparation.

Optionally, at the step (i) of processing the tissue sample, the method includes: slicing the tissue sample into fragments; incubating the fragments with one or more reagents in an assay plate under controlled conditions; and separating the tissue-resident extracellular vesicles from the tissue debris to obtain a tissue conditioned fluid. More optionally, each of the fragments weigh in a range of 0.01 to 0.25 milligram.

Optionally, the controlled conditions include an agitation in a range of 2 to 500 rotations per minute, at a temperature of 37° C. for a time period of 30 minutes, and filtering through a 70-micrometre filter.

Optionally, the one or more reagents are selected from a group of growth medium including a RPMI medium, proteases including a matrix metalloproteinase, collagenases including a collagenase D, and papain and nucleases including DNase 1, RNase, and Benzonase.

Optionally, at the step (b) of identifying the vesicle-associated proteins, the method includes: (i) analysing tissue-resident extracellular vesicles and lipoproteins; (ii) processing the isolated tissue-resident extracellular vesicles for isolating digested vesicle-associated peptides therefrom; (iii) separating and analysing the digested vesicle-associated peptides; and (iv) quantifying vesicle-associated proteins corresponding to the vesicle-associated peptides.

Optionally, at the step (e) of comparing the vesicle-associated protein profiles, the method includes: (a) obtaining the created vesicle-associated protein profiles for the identified vesicle-associated proteins in the tissue sample of the subject; (b) obtaining the pre-determined vesicle-associated protein profiles of the tumour tissue samples and/or non-tumour tissue samples associated with the presence or absence of colon cancer, or risk of developing colon cancer; and (c) checking if the created vesicle-associated protein profiles for the identified vesicle-associated proteins in the tissue sample of the subject matches with the pre-determined vesicle-associated protein profiles of the tumour tissue samples and/or non-tumour tissue samples.

Optionally, at the step (e) of comparing the vesicle-associated protein profiles, the method includes employing at least one of a nanoFCM analysis, ELISA, alphaLISA, FACS, fluorescent correlation microscopy and immune-electron microscopy.

Optionally, the quantified vesicle-associated proteins is selected from a group consisting of: FAP, MGAT5, ST6GAL1, SCD, CHST14, DPEP1, NUP210, NFXL1, CHPF2, CHSY1, FUT6, CERS6, GALNT6, MMP14, AGRN, ICAM1, SEL1L3, FAT1, FCGR1A, FKBP11, DDX46, GBP1, TMCO1, EPHB3, MME, NDC1, TMEM2, LILRB3, CYP4F3, STT3B, DNAJA1, TMEM214, TMEM63A, FUT8, TAPBP, EXT2, CERS2, GGCX, CEACAM5, RPL15, FUT4, GLCE, MAN2A2, RHBDF2, TMX2, ENTPD6, FAM57A, CHMP3, HM13, GPAA1, SEC62, and IKBIP.

Optionally, the vesicle-associated proteins is selected from a group consisting of: CD63, CD81, Flotillin-1, TSG101, FN1, collagen alpha-1 (XIII) chain (COL12Aa), prolyl endopeptidase fibroblast activating factor (FAP), DEFA1, PADI4, CHPF2, CHST14, GPA33, MMP14, TMEM2, CD9, Alix, TSG101, Annexin A5, CHMP1A, ICAM1, EPHB3-1, EPHB3-2, TMEM2-1, TMEM2-2,CHMP1B, CHMP2A, CHMP2B, CHMP3, CHMP4A, CHMP4A, CHMP5, CHMP6, RAB2A, RAB2B, RAB5A, RAB5B, RAB5C, RAB7A, RAB11B, RAB27A, RAB27B and RAB35.

According to a second aspect, the invention is realized by a system for identifying colon cancer-specific vesicle-associated proteins from a tissue sample of a subject, characterized in that the system includes:
(a) a kit for isolating tissue-resident extracellular vesicles from the tissue sample;
(b) a mass spectrometer for identifying vesicle-associated proteins associated with the isolated tissue-resident extracellular vesicles;
(c) a database having stored therein pre-determined vesicle-associated protein profiles of the tumour tissue samples and/or non-tumour tissue samples; and
(d) a computing unit in communication with the database, the computing unit including memory stored with executable codes operable to:
   (i) quantify the identified vesicle-associated proteins to identify one or more major vesicle-associated proteins,
   (ii) create vesicle-associated protein profiles for the identified vesicle-associated proteins, and
   (iii) compare the vesicle-associated protein profiles for the identified vesicle-associated proteins with the pre-determined vesicle-associated protein profiles of the tumour tissue samples and/or non-tumour tissue samples.

Optionally, the computing unit is further operable to obtain intensity information of the identified vesicle-associated proteins for quantification, by employing a labelling tool.

Optionally, the kit comprises: (a) a laboratory equipment for isolating the tissue-resident extracellular vesicles from a tissue sample; wherein the laboratory equipment comprises any of a surgical arrangement, an RT-PCR, test tubes, pipettes, assay plates, a centrifuge, a 70-micrometer filter, a density gradient, a quantification system, an electron microscope, an analyser, a mass spectrometer; (b) one or more reagents selected from a group consisting of: RPMI medium, DNase 1®, Collagenase D®, PBS medium, OptiPrep™ SDS, digestion buffer (trypsin/sodium deoxycholate), dithiothreitol, urea, TMT 11-plex isobaric mass tagging reagents®, TFA, ammonium formate buffer (formic acid), acetonitrile; (c) an epitope-specific binder against colon cancer-associated vesicle-associated proteins; and (d) at least one colon cancer-associated marker detection agent.

According to a third aspect, the invention is realized by a computer program product comprising non-transitory computer-readable storage medium having computer-readable instructions stored thereon, the computer-readable instructions being executable by a computerized device comprising processing hardware to execute aforesaid methods.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is an illustration of steps of a method of (namely, method for) identifying colon cancer-specific vesicle-associated proteins from a tissue sample of a subject, in accordance with an embodiment of the present disclosure;

FIG. 2A is an illustration of electron micrographs showing the vesicle isolates from tumour tissue samples and non-tumour tissue samples of three patients, in accordance with an embodiment of the present disclosure;

FIG. 2B is an illustration of depictions of tissue isolates in a tissue sample, in accordance with an embodiment of the present disclosure, in accordance with an embodiment of the present disclosure;

FIG. 2C is an illustration of data for particle measurement of the VAPs in tissue samples, in accordance with an embodiment of the present disclosure;

FIG. 2D is an illustration of a comparison graph between particles in a non-tumour and tumour tissue isolates in a tissue sample, in accordance with an embodiment of the present disclosure;

FIGS. 3A and 3B is an illustration of various charts to provide a proteomic overview of tissue-derived EVs, in accordance with an embodiment of the present disclosure;

FIGS. 4A to 4C are illustrations of various charts to show a quantitative difference of proteins in tissue-resident EVs derived from the tumour tissue samples, in accordance with an embodiment of the present disclosure;

FIG. 4D is an illustration of an enrichment analysis network generated using the commonly identified proteins, in accordance with an embodiment of the present disclosure;

FIG. 4E is an illustration of a chart to show KEGG pathways of lowest p-values associated with the VAPs present in tumour tissue-derived EVs, in accordance with an embodiment of the present disclosure;

FIG. 4F depicts the number of biomarker candidates identified in plasma, in accordance with an embodiment of the present disclosure;

FIGS. 5A to 5D are illustrations of various charts to show a quantitative difference of upregulated and downregulated proteins in tissue-resident EVs derived from the tumour tissue samples and non-tumour tissue samples, in accordance with an embodiment of the present disclosure;

FIG. 5E is an illustration of a bar graph depicting topology of the membrane proteins in tissue samples, in accordance with an embodiment of the present disclosure;

FIG. 5F is an illustration of depictions of tissue isolates, FAP and TMEM2, in a tissue sample, in accordance with an embodiment of the present disclosure;

FIG. 6 is an illustration of a flow chart depicting of steps of a method for sample preparation for identifying colon cancer-specific vesicle-associated proteins therefrom, in accordance with an embodiment of the present disclosure;

FIG. 7 is an illustration of a block diagram of a system for identifying colon cancer-specific vesicle-associated proteins from a tissue sample of a subject, in accordance with an embodiment of the present disclosure;

FIG. 8 is an illustration of depictions of Western blots for TMEM2 in tissue samples, in accordance with an embodiment of the present disclosure; and

FIG. 9 is a depiction of a proposed method to screen samples for the potential biomarkers, in accordance with an embodiment of the present disclosure.

In the accompanying diagrams, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

List of Abbreviations

| Abbreviation | Meaning |
|---|---|
| EVs | Extracellular vesicles |
| VAP | Vesicle-associated protein |
| VApt | Vesicle-associated peptide |
| VAPₘₐⱼ | one or more major Vesicle-associated protein |
| DNase 1 | Deoxyribonuclease 1 |
| RPMI medium | Roswell Park Memorial Institute medium |
| PBS | Phosphate-buffered saline |
| g | Gravitational constant |
| mL | Millilitre |
| nL | Nanolitre |
| mm | Millimetre |
| μm | Micrometre |
| mg | Milligram |
| min | Minute |
| MS | Mass spectrometry |
| LC-MS | Liquid chromatography-mass spectrometry |
| FASP | Filter-aided sample preparation |
| HCD | Higher energy collisional dissociation |
| SDS | Sodium dodecyl sulphate |
| TEAB | Tetra-ethyl-ammonium bromide |
| Ppm | Parts per-million moles |
| PE Signals | Pulsed electroacoustic signals |
| LFQ | Label free quantification |
| TMT | Tandem Mass Tag (a proteomic analysis) |
| TFA | Trifluoroacetic acid |
| CID | Collision-induced dissociation |

Definitions

As used herein, the following terms shall have the following meanings:

Throughout the present disclosure, the term “tissue-resident extracellular vesicle” or “extracellular vesicle”, “tissue-derived extracellular vesicles” or “EVs”, as used herein refers to nano-sized membrane-bound vesicles (particles or structures) released by a cell. Generally, EVs vary in size ranging from 20 nanometres (nm) to 10 microns (μm). Typically, the EVs consist of a liquid or cytoplasm enclosed by a membrane, often a lipid bilayer membrane with integrated proteins. EVs carry a cargo of proteins, nucleic acids, lipids, metabolites, toxins and often organelles from the parent cells to daughter cells. Furthermore, extracellular vesicles carry signatures of cells that produce them, i.e. parent cells, and thus reflect its phenotype, combined with an ability to spread to other parts of the body, such as circulating blood, thereby making EVs excellent candidates for biomarkers. Beneficially, the EVs may be used for therapeutic purposes, such as markers (namely, biomarkers) of disease, therapies of disease (namely, by delivering cargo to an affected tissue), infer a cell's origin (such as a tumour cell) and so on. Moreover, the role of EVs as biomarkers is associated with the release and uptake of EVs by cells to potentially induce a response in the recipient cell. Furthermore, the EVs consist of specifically selected components which can confer functions in recipient cells, making them interesting from a treatment perspective as well. Optionally, EVs may also be referred to as exosomes, ectosomes, microvesicles, microparticles, prostasomes, oncosomes, membrane fragments, plasma membrane vesicles, inclusion vesicles or apoptotic bodies, depending upon cellular origin thereof. For example, ectosome, microvesicle and microparticle refer to particles released from the surface of cells, and apoptotic bodies are released by cells undergoing apoptosis. Typically, EVs are found in bodily fluids such as blood (plasma and serum), urine, saliva, cerebrospinal fluid, semen, ascites, synovial fluid, bronchoalveolar lavage, pleural effusion, amniotic fluid, sweats, feces, cystic fluids, tears and breast milk; tissues; or cells.

The term “membrane” as used herein refers to a biological membrane, i.e. an outer covering of cells and/or organelles that allow passage of certain compounds. Typically, membrane of the present disclosure refers to the EV membranes (namely, covering of EVs) which encloses an organellar content as EV or organelle, and which opens to release the enclosed organellar content therefrom. Such membrane are a single lipid layer or lipid bilayers with integrated proteins to protect the inner content thereof. Moreover, the membranes may originate from the outer cell membrane, the Golgi-apparatus, the Endoplasmic reticulum, the nucleus or the mitochondria.

As used herein, the term “lipoproteins” refers to a stabilized biochemical assembly for transferring compounds from one site of a cell to another site of the cell or other cells (namely cell to cell communication). In an embodiment, lipoproteins transport hydrophobic lipid molecules, such as triacylglycerols, phospholipids and cholesterol, in extracellular water of the body to all cells and/or tissues, such as blood plasma. Examples of lipoproteins include various enzymes, transporters, structural proteins, adhesins, antigens and toxins.

As used herein, the term “biomarker” or “marker” refers to a measurable biological indicator that corresponds to a presence or progression of an underlying biological condition or disease. Generally, the biomarkers are proteins, genes, RNAs (including microRNAs, mRNAs and so on), DNA, peptides, lipids, circulating structures such as vesicles, and variations or modifications thereof.

As used herein, the term, “tissue sample” refers to an aggregate of similar cells and extracellular matrix thereof with a specific function. Typically, the tissue sample may include an affected tissue (such as a tumour tissue sample) or a normal tissue (such as a non-tumour tissue sample). In an example, a tumour sample is a colon cancer tissue sample derived from biopsy of a subject, and a non-tumour tissue sample may be a tissue sample derived from a membrane, preferably located at a distance of around 10 cm from the tissue sample. The tissue sample may be a fresh tissue sample (namely, isolated at the time of testing) or a frozen tissue sample (namely, a preserved sample for subsequent research). Beneficially, the tissue-derived EVs, namely tissue-resident EVs (hereinafter referred to as “EVs”), represent closely the tumour microenvironment with the cancer secretome is at its peak concentration, thus eliminating a potential bias from the non-tumour entities, such as in circulation and/or any cell line counterpart. Moreover, the tissue samples comprise a subpopulation of EVs that is enriched in mitochondrial proteins and such subpopulation may be further identified in circulation of cancer patients. Thus, EVs may directly be isolated from the tissue interstitium and subsequently the protein cargo is analysed by quantitative mass spectrometry to search for potential biomarkers for colon cancer. As control, macroscopically normal colonic mucosa is sampled, hereafter referred to simply as non-tumour tissue sample. Alternatively, cancer cell lines as model systems or body fluid-derived EVs may be used as samples from the subjects.

As used herein, the term “subject” refers to an individual or a participant, a human or an animal or a mammal, subjected to a research study, a treatment or an observation. The subject includes a patient diagnosed with a disease, a person suffering from one or more symptoms of a disease (undiagnosed), or a normal person with no disease symptoms, and for receiving an investigational product(s) or as a control (namely, receiving a placebo). Typically, the participant is included in a study subsequent to their consent to the research. However, where the subject is unable to submit their consent, such as for example a baby or an animal, a guardian thereof is required to submit consent on their behalf.

As used herein, the term “colon cancer-specific vesicle-associated proteins” or “vesicle-associated proteins” or “VAP” refers to immunological modulators that are derived from, or are a part of, the EVs, such as for example biological membranes of EVs. The VAPs may include, but are not limited to, integral VAPs and peripheral VAPs, based on their localization in the EVs. Typically, the VAPs are specific for specific tissues and thus are promising biomarkers for diseases associated with such tissues. For example, VAPs are indicative of the colon cancer in a subject. The VAPs include, but do not limit to, CD9, CD63, and CD81, as discussed in more details in description of embodiments of the present disclosure.

As used herein, the term “digested vesicle-associated peptides” or “vesicle-associated peptides” or “VApt” refers to smaller chains of amino acids, such as two or more amino acids, preferably two to fifty amino acids, held together by peptide bonds. In an embodiment, the VAPs are digested into shorter fragments using digesting enzymes to produce VApt.

The term “one or more major vesicle-associated proteins” or “VAPₘₐⱼ” as used herein refers to specific VAPs that code for a specific disease, such as colon cancer. The VAPₘₐⱼ may individually code for a disease and are generally easily identifiable.

As used herein, the term “profiling” refers to identifying intensity-related information of the VAPₘₐⱼ and plotting the intensity-related information of the VAPₘₐⱼ in respect of the intensity-related information of the VAPs derived from tumour tissue samples and/or non-tumour tissue samples. The term profiling may also relate to identifying a biomarker associated with colon cancer.

As used herein, the term “vesicle-associated protein profiles” or “VAP profiles” refers to an intensity-related information of the VAPₘₐⱼ.

As used herein, the term “pre-determined vesicle-associated protein profiles” refers to an intensity-related information of the VAPs derived from tumour tissue samples and/or non-tumour tissue samples.

The terms “cell media”, “culture media” and/or “cell culture media” as used herein refer to a growth media used for preserving or culturing tissue samples, cells and/or cell lines obtained from the subject during surgery. The culture media include supplements required for culturing and preserving carcinoma cell lines. The culture media may be an ordinary medium or may also be liquid nitrogen-based medium. The culture medium may be isotonic, hypotonic, or hypertonic depending upon the type of tissue samples, cells and/or cell lines. In an embodiment, the culture medium may contain a buffer and/or at least one salt or a combination of salts. Moreover, the buffer functions as pH-stabilizing agents and may maintain pH within a particular range, for example, between 1 and 12, preferably in a range of 8 to 9.

The term “database” as used herein refers to an organized body of digital information regardless of a manner in which the data or the organized body thereof is represented. More optionally, the database may be hardware, software, firmware and/or any combination thereof. For example, the organized body of digital information may be in a form of a table, a map, a grid, a packet, a datagram, a file, a document, a list or in any other form. The database includes any data storage software and system, such as, for example, a relational database like IBM DB2 and Oracle 9.

The term “computing unit” as used herein refers to at least one programmable or computational entity configured to perform specific tasks associated with the system. Specifically, the computing unit is configured to host computer programs and/or routines that are operable to perform specific tasks associated with the system. It will be appreciated that the specific tasks performed by the system refers to acquiring, storing, identifying and processing information to achieve the goal of the system. Optionally, the computing unit can be a single computational entity and/or plurality of computational entities operating in a parallel or distributed architecture to perform the specific tasks associated to the system. Optionally, the computing unit can be implemented as a computer program that provides various services (such as database service) for the system.

### DETAILED DESCRIPTION

The practice of the embodiments described in further detail below will employ, unless otherwise indicated, conventional methods of diagnostics, molecular biology, cell biology, biochemistry and immunology within the skill of the art. Such techniques are explained fully in the literature.

It is appreciated that certain features of the invention, which are for clarity described in the context of separate embodiments, may also be provided in combination in a single embodiment. Conversely various features of the invention which are for brevity described in the context of a single embodiment, may also be provided separately and/or in any suitable sub-combination.

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In overview, embodiments of the present disclosure are concerned with a method of identifying colon cancer-specific vesicle-associated proteins from a tissue sample of a subject. Furthermore, embodiments of the present disclosure are concerned with a system for identifying colon cancer-specific vesicle-associated proteins from a tissue sample of a subject.

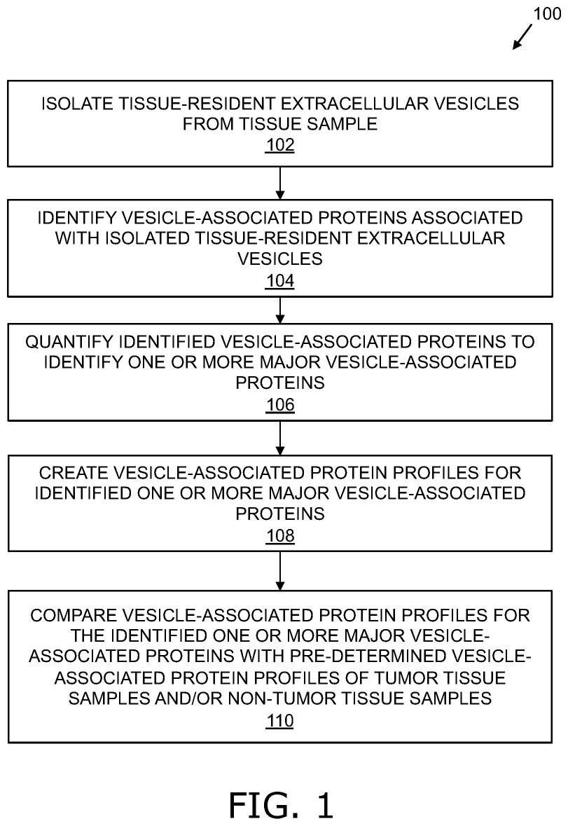

As illustrated in FIG. 1, there are shown steps of a method 100 of (namely, a method for) identifying colon cancer-specific vesicle-associated proteins from a tissue sample of a subject, in accordance with an embodiment of the present disclosure. The method comprises, at a step 102, isolating tissue-resident extracellular vesicles from the tissue sample; at a step 104, identifying vesicle-associated proteins associated with the isolated tissue-resident extracellular vesicles; at a step 106, quantifying the identified vesicle-associated proteins to identify one or more major vesicle-associated proteins; at a step 108, creating vesicle-associated protein profiles for the identified vesicle-associated proteins; and at a step 110, comparing the vesicle-associated protein profiles for the identified vesicle-associated proteins with pre-determined vesicle-associated protein profiles of the tumour tissue samples and/or non-tumour tissue samples.

It will be appreciated that the present disclosure is related to pre-determining vesicle-associated protein profiles specific to cancer, specifically colon cancer (namely, colorectal cancer) and further provides a diagnostic tool for colon cancer operable to identify biomarkers in a biological sample from a subject diagnosed with colon cancer or at a risk of potentially developing colon cancer. Biomarkers associated with the colon cancer are present in the extracellular vesicles (EVs), namely tissue-resident EVs. Moreover, EVs contain information of the originating cells and therefore EVs derived from the tumour tissue samples are potential candidates for identification of specific conditions or diseases. The EVs can be isolated, and later analysed (namely, profiled) for cargo thereof, from one or more biological samples from the subject (for example blood, plasma, serum, urine, saliva, spinal fluid, tissues, breast milk, bone marrow, amniotic fluid, and so forth). Moreover, the EV cargo, such as peptides, proteins, nucleic acids, lipids, and modifications thereof, are suitable for identifying diseases or specific conditions. Furthermore, isolation and detection of specific biomarkers rely significantly on obtaining samples from tissues to identify the specific condition or disease. Notably, the specific biomarkers, namely extracellular vesicles-associated biomarkers, are produced in low or moderate amounts in normal tissues, for example non-tumour tissue samples (such as normal mucosal membrane), other than an affected tissue samples, for example tumour tissue samples, thus lacking specificity. The EVs and EV-associated biomarkers for colon cancer, namely colon cancer-specific vesicle associated proteins (VAPs) are identified, quantified and profiled.

Optionally, the profiled colon cancer-specific VAPs, namely pre-determined VAP profiles, are stored in a database for future references for inferring whether or not a tissue sample from a subject is an affected tissue or a healthy tissue and the risks of potential development of the specific conditions or disease associated therewith.

Optionally, the tissue sample is a tumour tissue sample and/or a non-tumour tissue sample. More optionally, the tumour tissue sample is an affected tissue localised in an affected organ, such as for example colon, rectum or any other part of the large intestine. Furthermore, optionally, the tumour tissue sample is surgically removed from a subject, for example using specialist sample collecting apparatus that is inserted into the colon, for example along the colon. Optionally, the non-tumour tissue sample is a macroscopically normal appearing mucosa (namely, normal mucosa). More optionally, the non-tumour tissue sample is collected from a site 10 cm from the tumour of the subject. Furthermore, optionally, the tumour-tissue sample and/or the non-tumour tissue sample are obtained as a fresh sample from the subject during the tumour resection surgery, or alternatively used as a frozen sample thereof. More optionally, the tumour-tissue samples and the non-tumour tissue samples are collected from ten patients, thereby resulting in a total of 20 samples from which EVs are required to be isolated. Moreover, the tissue samples are collected from both male and female subjects, i.e. patients. Furthermore, the age limit of the subjects is in a range of 56 to 87 years old. Table 1 provides the patient demographics.

TABLE 1
Patient demographics

| Patient | Sex | TNM-stage |
|---|---|---|
| 1 | Male | pT2N0 |
| 2 | Female | pT3bN2a |
| 3 | Female | pT3N0 |
| 4 | Female | pT4aN1b |
| 5 | Female | pT3bN0 |
| 6 | Male | T3bN0 |
| 7 | Female | T2N0 |
| 8 | Female | T3cN2a |
| 9 | Female | T3dN0 |
| 10 | Female | T4aN0 |

In an embodiment, at the step 102 of the method 100, the method 100 includes processing the tissue sample to obtain a tissue conditioned fluid. Typically, processing of the tissue sample comprises obtaining the tissue sample and culturing (namely, growing and/or preserving) the tissue sample. The tissue sample is surgically removed from the subject. It will be appreciated that the tissue sample of the subject is removed surgically by an authorized professional, such as a doctor or researcher alone or along with a one or more research nurse, under sterile conditions.

Optionally, at the step (i) of processing the tissue sample, the method includes: slicing the tissue sample into fragments; incubating the fragments with one or more reagents in an assay plate under controlled conditions; and separating the tissue-resident extracellular vesicles from the tissue debris to obtain a tissue conditioned fluid. Optionally, each of the fragments weigh in a range of 0.01 to 0.25 milligram (mg). Specifically, the tissue pieces are weighed and then divided into pieces of 0.01 to 0.25 mg, preferably 0.2 mg pieces, each of which is placed in a well of the assay plate, for example a 6-well plate containing one or more reagents. Optionally, the one or more reagents are selected from a group of growth medium including a RPMI medium, proteases including a matrix metalloproteinase, collagenases including a collagenase D, and papain and nucleases including DNase 1, RNase, and Benzonase. It will be appreciated that a same ratio of the tissue sample (weight):one or more reagents (volume/concentration) is maintained for processing the tissue samples. Moreover, the tissue sample is divided in 0.2 mg pieces for the convenience to work with. Furthermore, the tissue is further minced into approximately 2×2 mm² pieces before the actual enzymatic treatment to make sure that enzymes can easily permeate the tissue sample. In an example, approximately 0.2 mg fragments of tissue samples, generally 2×2 mm² (or 2×2×0.25 mm³ or 1 mm³) in size, are incubated in 2 mL of growth medium, for example RPMI 1640 medium or RPMI medium® (Sigma-Aldrich), at a temperature of 37° C. for 30 minutes. Notably, the RPMI medium is a growth medium formulated to support lymphoblastoid cells, leukemia cells and a wide variety of cells that are anchorage dependent, in a suspension culture. Optionally, the RPMI medium contains DNase 1® (Roche, Basel, Switzerland) having a concentration of 2 milligram/millilitre (mg/mL) and Collagenase D® (Roche) having a concentration of 40 unit/millilitre (U/mL). After incubation, the tissue samples are passed through a filtration arrangement, and cell debris are eliminated or segregated by centrifugation. Optionally, the filtration arrangement is a 70-micrometre (μm) filter. Additionally, the 6-well plate is further rinsed with 1 mL of fresh RPMI medium® which was also passed through the filter. The resulting filtered liquid, from the tissue samples as well as the 6-well plate, now void of visible tissue debris, is carried over for isolation of tissue-resident EVs. Furthermore, optionally, the controlled conditions include an agitation in a range of 2 to 500 rotations per minute (rpm), preferably in a range of 10 to 50 rpm, more preferably 20 rpm at a temperature of 37° C. for a time period of 30 minutes, and filtering through a 70-micrometre filter.

In an embodiment, at the step 102 of the method 100, the method 100 includes collecting the tissue-resident extracellular vesicles from the tissue conditioned fluid. The tissue-resident EV isolation follows a centrifuge-based protocol. In this regard, the tissue conditioned fluid is centrifuged at a speed of 300×g for a time period of 10 minutes to remove any potentially contaminating cells therefrom. Subsequently, the supernatant is re-centrifuged for collecting small and large EVs. In this regard, the supernatant is sequentially centrifuged at a speed of: (i) 2,000×g_{avg} for 20 minutes to separate very large EVs (such as apoptotic bodies) and remove any cell debris; (ii) 16,500×g_{avg} for 6 minutes (TLA 100.3, k-factor: 404.5, Beckman Coulter ultracentrifuge) to separate large EVs; and (iii) 120,000×g_{avg} for 65 minutes (TLA 100.3, k-factor: 55.5, Beckman Coulter ultracentrifuge) to pellet smaller EVs. The collected small and large EVs pellet are suspended in a PBS solution for disengaging cells clumped with the aforementioned small and large EVs.

In an embodiment, at the step 102 of the method 100, the method 100 includes purifying the collected tissue-resident extracellular vesicles by a density gradient preparation. The collected small and large EVs pellets are further purified by an isopycnic centrifugation. Typically, the isopycnic centrifugation separates the small and large EVs on the basis of their density by establishing a density gradient, for example by using Iodixanol Density Cushion™ (OptiPrep™ density gradient, Sigma Aldrich). In this regard, the 1 mL of small and large EVs is mixed with 3 mL of 60% OptiPrep™ (or Iodixanol), and bottom-loaded in an ultracentrifuge tube. Subsequently, 4 mL of 30% OptiPrep™ and 4 mL of 10% of OptiPrep™ is layered on top of the aforementioned mixture bottom-loaded in the ultracentrifuge tube. The density gradient arrangement is subjected to ultracentrifugation at 97,000×g_{avg} for 2 hours (SW 41 Ti Swinging Bucket Rotor for use in Beckman Coulter ultracentrifuge, k-factor: 265.1) for extracting vesicle-associated proteins from the EVs. A visible band between the 10% OptiPrep™/30% OptiPrep™ interface is collected (namely, at the intersection of 1.078 g/mL and 1.175 g/mL OptiPrep™), preferably with a pipette. The collected visible band comprises the purified vesicle-associated proteins. Typically, the isolation of tissue-resident EVs includes separating the EVs from lipoproteins. Beneficially, the Iodixanol Density Cushion™ allows EVs to float and lipoproteins to float on top thereof. The VAPs are subsequently isolated from the EVs and analysed with mass spectrometry for identifying the VAPs.

In an embodiment, VAPs is separated by sodium dodecyl sulfate-polyacrylamide gel electrophoresis (SDS-PAGE) on a precast polyacrylamide gel (Mini-Protean@ TGX™ 4-12% gel, Bio-Rad Laboratories, Hercules, Calif., USA). The gel is transferred onto polyvinylidene fluoride (PVDF) membranes for detecting high molecular weight proteins, in this case VAPs. The aforesaid transfer is performed using the Trans-Blot@ Turbo™ Transfer System (Bio-Rad), where PVDF membranes are blocked for two hours in a blocking buffer, TBST (comprising a mixture of tris-buffered saline (TBS) and polysorbate 20 (also known as Tween 20)) containing 5% non-fat dry milk. Subsequently, the PVDF membranes are probed with primary antibodies against flotillin-1 (such as anti-Flotillin-1, Abcam ab133497, clone EPR6041), CD81 (such as anti-CD81, Abcam ab79559, clone M38), CD63 (BD Pharmingen 556019, clone H5C6), FAP (anti-FAP, R&D Systems AF3715) and TMEM2 (anti-TMEM2, Sigma-Aldrich, HPA044889) diluted 1,000 times in blocking buffer over-night at 4° C. The PVDF membranes are washed three times in TBST and then probed with horseradish peroxidase conjugated secondary antibodies diluted 10,000 times in blocking buffer for one hour at room temperature. The PVDF membranes are then washed an additional three times in TBST before addition of substrate SuperSignal™ West Femto Maximum Sensitivity Substrate (Thermo Fisher Scientific) and analysed using a ChemiDoc™ Imaging System, (Bio-Rad).

In an embodiment, at the step 104 of the method 100, the method 100 includes identifying vesicle-associated proteins associated with the isolated tissue-resident extracellular vesicles. Typically, identifying the VAPs associated with the isolated tissue-resident extracellular vesicles that are absent in the non-tumour tissue samples serve as excellent candidates for biomarkers for colon cancer. Generally, conventional analysis tools, including, but not limited to electron microscopy, protein assays (for example Qubit Assay™ Protein Assay Kit (Thermo Fisher Scientific)), nanoparticle tracking analysis and/or ExoView analysis may be applied, according to manufacturer's instructions, for identifying and quantifying the extracted VAPs. Typically, the isolation of tissue-resident EVs includes separating the tissue-resident EVs from the lipoproteins. More optionally, the method includes processing and analysing tissue-resident extracellular vesicles and lipoproteins, optionally, in two sets. The tissue samples, comprising tumour tissue samples and non-tumour tissue samples, were subdivided into two Tandem Mass Tag (TMT) proteomic analysis sets, Set 1 and Set 2, comprising over 2500 quantified proteins (as discussed in detail below), wherein the Set 2 comprises more proteins compared to the Set 1.

Optionally, at the step 104 of the method 100, the method 100 includes: (i) analysing tissue-resident extracellular vesicles and lipoproteins; (ii) processing the isolated tissue-resident extracellular vesicles for isolating digested vesicle-associated peptides therefrom; (iii) separating and analysing the digested vesicle-associated peptides; and (iv) quantifying vesicle-associated proteins corresponding to the vesicle-associated peptides. The method further includes processing the isolated tissue-resident EVs for isolating the digested vesicle associated peptides (VApt) associated with the isolated tissue-resident EVs. The tissue-resident EVs are digested with trypsin using the filter-aided sample preparation (FASP) method. Briefly, 50 μg of the tissue-resident EVs sample is lysed by the addition of sodium dodecyl sulphate (SDS) to a final concentration of 2%. The method further includes reducing the tissue-resident EVs sample with 100 mM dithiothreitol at 60° C. for 30 minutes, transferring the reduced samples on to a 30 kDa MWCO™ Pall Nanosep® Centrifugation Filters (Sigma Aldrich), washing the filtered sample first with 8 M urea solution and later with digestion buffer, comprising 1% sodium deoxycholate (SDC) in 50 mM TEAB, prior to alkylation with 10 mM methyl methanethiosulfonate in digestion buffer for 30 minutes (min). Thereafter, a further digestion of tissue-resident EVs sample is performed in the digestion buffer at 37° C. in two stages: the samples are incubated with 0.5 μg of Pierce™ MS-grade trypsin (Thermo Fisher Scientific) overnight, and thereafter the samples are incubated with an 0.5 μg of trypsin for 2 hours. Subsequently, the VApt are collected by centrifugation. Furthermore, the digested VApt are labelled using TMT 11-plex™ isobaric Mass Tagging Reagents (Thermo Fisher Scientific) according to the manufacturer instructions and SDC was removed by acidification with 10% TFA. The combined samples were pre-fractionated with basic reversed-phase chromatography (bRP-LC) using a Dionex UltiMate™ 3000 UPLC system (Thermo Fisher Scientific).

The method further includes separating the digested VApt by suspending in a Solvent A comprising 10 mM ammonium formate buffer at pH 10.00, and performing a basic reversed-phase chromatography (bRP-LC) using a Dionex UltiMate™ 3000 UPLC system. The peptide (namely, VApt) separations are beneficially performed on a reversed-phase XBridge BEH C18 column (3.5 μm bead size, 3.0×150 mm, Waters Corporation). The reversed-phase XBridge BEH C18 column is loaded with the VApt and UPLC gradient comprising Solvent A and a linear gradient of Solvent B comprising 90% acetonitrile and 10% 10 mM ammonium formate at pH 10.00, from 3% to 40% Solvent B over 17 min followed by an increase to 100% Solvent B over 5 min at a flow rate of 0.4 mL/min for separating the VApt. The VApt are concatenated into 20 VApt, dried and reconstituted in 3% acetonitrile, 0.2% formic acid.

The method further includes quantifying VAPs corresponding to the VApt. Each VApt is, for example, analysed on an Orbitrap Fusion™ Tribrid™ mass spectrometer interfaced with Easy-nLC™ 1200 nanoflow liquid chromatography system (Thermo Fisher Scientific). VApt are trapped on an Acclaim™ Pepmap™ 100 C18 trap column (100 μm×2 cm, particle size 5 μm, Thermo Fisher Scientific) and separated on an in-house packed analytical column (75 μm×300 mm, particle size 3 μm, Reprosil-Pur C18, Dr. Maisch) using a mobile phase containing a Solvent A comprising 0.2% formic acid and Solvent B comprising 80% acetonitrile in 0.2% formic acid. The Solvent B forms a linear gradient from 6% to 32% Solvent B over 75 min followed by an increase to 50% Solvent B over 5 min and then 100% Solvent B for 5 min at a flow rate of 300 nL/min. Thereafter, precursor ion mass spectra are acquired at a resolution of 120,000 and Tandem Mass Spectroscopy (MS/MS) analysis is performed in a data-dependent multi-notch mode where CID spectra of the most intense precursor ions are recorded in ion trap at collision energy setting of 30 for 3 s (‘top speed’ setting). Charge states 2 to 7 are selected for fragmentation, dynamic exclusion is set to 45 s and 10 ppm. MS3 spectra for reporter ion quantitation are recorded at 60,000 resolution with HCD fragmentation at collision energy of 55 using the synchronous precursor selection. The MS/MS analysis of the tissue-resident EVs sample provides the VAPs (as listed in Table-2 below) detectable in tumour (colon cancer) tissue samples but not in non-tumour tissue samples. The detected VAPs may then be used in the method 100 for identifying colon cancer-specific VAPs.

Moreover, the isolation and analysis of the tissue-resident EVs and the VAPs associated with the isolated tissue-resident EVs were conducted in two sets, namely Set 1 and Set 2. The Set 1 and Set 2 comprises a total of 10 healthy samples and 10 tumour tissue paired samples from 10 patients for proteomic analysis. Samples from 5 patients are processed and analysed separately from the samples from the other 5 patients, thus resulting in two sets, Set 1 and Set 2, respectively. The VAPs associated with the isolated tissue-resident EVs as identified by any of the aforementioned methods yielded in a total of 2567 and 3742 quantified VAPs for Set 1 and Set 2, respectively as shown in Table 2. Table 2 provides common VAPs associated with the isolated tissue-resident EVs and referred to as EV markers, as well as other potentially relative VAPs that are associated with EVs and their biogenesis. An ‘X’ demarks that a corresponding EV marker is successfully identified in the dataset. Notably, several hundred VAPs were reported to be differently expressed in EVs derived from tumour-tissue samples as compared to the EVs from non-tumour tissue samples. Furthermore, 2271 VAPs were reported in both the sets, however, 155 out of 2271 VAPs were found to be significantly dysregulated in tumour-tissue samples. Common EV markers such as CD63, TSG101 and Flotillin 1, amongst others, were identified in both the sets. Likewise, other relevant proteins such as components of the ESCRT machinery as well as a number of Rab proteins were among the identified proteins as shown in Table 2.

TABLE 2
Common VAPs associated with the tissue-resident EVs

| | Protein | Set 1 | Set 2 |
|---|---|---|---|
| Vesicle markers | CD9 | | X |
| | CD63 | X | X |
| | CD81 | X | X |
| | Flotillin 1 | X | X |
| | Alix | X | X |
| | TSG101 | X | X |
| | Annexin A5 | X | X |
| ESCRT proteins | CHMP1A, B | X | X |
| | CHMP2A, B | X | X |
| | CHMP3 | X | X |
| | CHMP4A | | X |
| | CHMP4B | X | X |
| | CHMP5 | X | X |
| | CHMP6 | X | X |
| | VPS4A, B | X | X |
| | VPS25 | | X |
| | HGS | X | X |
| RAB's | RAB7A | X | X |
| | RAB11B | X | X |
| | RAB27A | X | X |
| | RAB27B | | X |
| | RAB2A | X | X |
| | RAB2B | | X |
| | RAB5A, B, C | X | X |
| | RAB11B | X | X |
| | RAB35 | X | X |

In an embodiment, at the step 106 of the method 100, the method 100 includes quantifying the identified vesicle-associated proteins to identify one or more major vesicle-associated proteins. Optionally, the method 100 includes quantifying the identified VAPs to identify the one or more major vesicle-associated proteins (VAPₘₐⱼ). The method 100 may include searching the identified VAPs in respect of the pre-determined VAP profiles of the tumour tissue samples and/or non-tumour tissue samples, identifying the one or more VAPₘₐⱼ for each group of the identified VAPs, and obtaining intensity information relating to the one or more VAPₘₐⱼ by employing a label-free quantification tool or an isotope labeling-based quantification tool. For example, searching the identified VAPs may be performed on the basis of at least one parameter including an enzyme specificity, a variable modification, a fixed modification, oxidation properties, carbamidomethylation properties and an ion tolerance. Moreover, the information relating to the intensity of the one or more VAPₘₐⱼ may include, but is not limited to, the concentration and/or sequence lengths of the one or more VAPₘₐⱼ. For example, the one or more VAPₘₐⱼ may be identified by searching the VAPs on the Andromeda search engine on the basis of at least one parameter including an enzyme specificity, trypsin; a variable modification, an oxidation of methionine (15.995 Da); a fixed modification, carbamidomethylation of cysteine (57.021 Da); two missed cleavages; 20 ppm for precursor ions tolerance and 4.5 ppm for fragment ions tolerance; Homo sapiens reference proteome data from Swiss-Prot (20,196 entries); 1% false discovery rate; and a minimum peptide length of seven amino acids. Thereafter, a first VAPₘₐⱼ is identified and chosen as a representative VAP of each group. The representative VAP is further used for identifying the intensity related information of the one or more VAPₘₐⱼ.

It will be appreciated that the above steps 102 to 106 of the method 100 are common and could be used for identifying the colon cancer-specific VAPs in tissue samples from subjects. In this regard, a tissue sample, namely tumour tissue sample and/or non-tissue sample, is used to isolate EVs therefrom, and identify and quantify VAPs associated with the isolated EVs. Moreover, the steps could also be used for diagnosing colon cancer and/or a potential risk of development of colon cancer in a subject. In this regard, the VAPs identified and quantified from the isolated EVs using the aforesaid steps, are used to create a database comprising a list of all the VAPs identified as biomarker candidates for colon cancer. It will be further appreciated that the database comprises a different list of VAPs, namely pre-determined VAP profiles, for each of the VAPs associated with the isolated tissue-resident EVs derived from tumour tissue samples and VAPs associated with the isolated tissue-resident EVs derived from non-tumour tissue samples.

In an embodiment, at the step 108 of the method 100, the method 100 includes creating vesicle-associated protein profiles for the identified one or more major vesicle-associated proteins. Optionally, the data files for each set are merged for identification and relative quantification using Proteome Discoverer™ version 2.2 (Thermo Fisher Scientific). Identification and relative quantification are performed with Proteome Discoverer™ version 2.2. The search is against Homo sapiens Swissprot Database (Nov 2017) using Mascot™ 2.5 (Matrix Science) as a search engine with precursor mass tolerance of 5 ppm and fragment mass tolerance of 0.6 Da. Tryptic peptides (trypisin-digested VApt) are accepted with zero missed cleavage, variable modifications of methionine oxidation and fixed cysteine alkylation, TMT-label modifications of N-terminal and lysine are selected. Percolator algorithm is used for the validation of identified VAPs and the quantified VAPs are filtered at 1% FDR and grouped by sharing the same sequences to minimize redundancy. Only VApt unique for a given VAP are considered for quantification of the VAP and ratios are calculated by dividing the samples with the reference sample. Consequently, the resultant data is referred to and used as the vesicle-associated protein profiles for the identified one or more major vesicle-associated proteins.

Optionally, the method 100 may include creating the pre-determined VAP profiles of the tumour tissue samples and/or non-tumour tissue samples. For example, the method 100 may include isolating tissue-resident EVs from the tumour tissue samples and/or non-tumour tissue samples, identifying the VAPs associated with the isolated tissue-resident EVs, and quantifying the identified VAPs for creating the pre-determined VAP profiles of the tumour tissue samples and/or non-tumour tissue samples.

In an embodiment, at the step 110 of the method 100, the method 100 includes comparing the vesicle-associated protein profiles for the identified one or more major vesicle-associated proteins with pre-determined vesicle-associated protein profiles of the tumour tissue samples and/or non-tumour tissue samples. Optionally, the method includes detecting biomarkers in VAPs of the tissue-resident EVs of the subject by comparing the tissue-resident EVs derived from tumour samples with tissue-resident EVs derived from non-tumour samples used as control. Optionally, at the step 110 of the method 100, the method 100 includes: (a) obtaining the created vesicle-associated protein profiles for the identified vesicle-associated proteins in the tissue sample of the subject; (b) obtaining the pre-determined vesicle-associated protein profiles of the tumour tissue samples and/or non-tumour tissue samples associated with the presence or absence of colon cancer, or risk of developing colon cancer; and (c) checking if the created vesicle-associated protein profiles for the identified vesicle-associated proteins in the tissue sample of the subject matches with the pre-determined vesicle-associated protein profiles of the tumour tissue samples and/or non-tumour tissue samples. It will be appreciated that the matching confirms a presence or an absence of colon cancer-specific VAPs in the tissue samples, thereby diagnosing the subject with a potential colon cancer, or risk of developing colon cancer.

Optionally, the method 100 employs at least one of a nanoFCM analysis, ELISA, alphaLISA, FACS, fluorescent correlation microscopy and immune-electron microscopy, or other methods for detecting VAPs associated with the isolated tissue-resident EVs. The nanoFCM analysis is performed for plotting the created VAP profiles for the identified one or more VAPₘₐⱼ with the pre-determined VAP profiles of the tumour tissue samples and/or non-tumour tissue samples. The analysis using at least one of the aforementioned techniques enables identifying numerous VAPs upregulated in tumour tissue samples as compared to the non-tumour tissue samples. Further analysis using conventional Bioinformatics tools showed different pathways to be dominant in the EV proteome in tumour tissue samples as compared to the non-tumour tissue samples, with protein biosynthesis being significantly upregulated in the tumour tissue samples, and relatively higher expression of energy production pathways in non-tumour tissue samples.

Optionally, the quantified vesicle-associated proteins is selected from a group consisting of: FAP, MGAT5, ST6GAL1, SCD, CHST14, DPEP1, NUP210, NFXL1, CHPF2, CHSY1, FUT6, CERS6, GALNT6, MMP14, AGRN, ICAM1, SEL1L3, FAT1, FCGR1A, FKBP11, DDX46, GBP1, TMCO1, EPHB3, MME, NDC1, TMEM2, LILRB3, CYP4F3, STT3B, DNAJA1, TMEM214, TMEM63A, FUT8, TAPBP, EXT2, CERS2, GGCX, CEACAM5, RPL15, FUT4, GLCE, MAN2A2, RHBDF2, TMX2, ENTPD6, FAM57A, CHMP3, HM13, GPAA1, SEC62, and IKBIP. More optionally, the quantification is performed by employing at least one of the aforementioned techniques, i.e. nanoFCM analysis, ELISA, alphaLISA, FACS, fluorescent correlation microscopy and immune-electron microscopy, or other methods for detecting VAPs associated with the isolated tissue-resident EVs. Approximately 52 different VAPs are susceptible to being identified to be significantly upregulated in colon cancer, and thus may serve as potential biomarker candidates in colon cancer. Table 3, provided as an Appendix to Specification, lists the UniProt IDs and short description corresponding to the aforementioned quantified 52 VAPs.

Optionally, the vesicle-associated proteins is selected from a group consisting of: CD63, CD81, Flotillin-1, TSG101, FN1, collagen alpha-1 (XIII) chain (COL12Aa), prolyl endopeptidase fibroblast activating factor (FAP), DEFA1, PADI4, CHPF2, CHST14, GPA33, MMP14, TMEM2, CD9, Alix, TSG101, Annexin A5, CHMP1A, ICAM1, EPHB3-1, EPHB3-2, TMEM2-1, TMEM2-2,CHMP1B, CHMP2A, CHMP2B, CHMP3, CHMP4A, CHMP4A, CHMPS, CHMP6, RAB2A, RAB2B, RAB5A, RAB5B, RAB5C, RAB7A, RAB11B, RAB27A, RAB27B and RAB35. It will be appreciated that the aforesaid VAPs serve as potential biomarkers for colon cancer as evident from their significant upregulation and/or downregulation, and may be used in the clinical setting.

In an embodiment, the EVs are directly isolated from the tissue samples surgically removed from the subject, i.e. a patient's tumour tissue samples. It will be appreciated that the concentration of the tissue-resident EVs, i.e. the EVs released specifically by the cancerous cells, exists in the highest concentration in such tumour tissue samples, and such tissue-resident EVs best reflect a snapshot of the vesicular secretome of the tumour tissue samples. Moreover, the tissue-resident EVs and vesicle-associated proteins derived therefrom substantially reflect the in-vivo environment of the tumour tissue samples, thereby offering a far more relevant sample than that of cell lines.

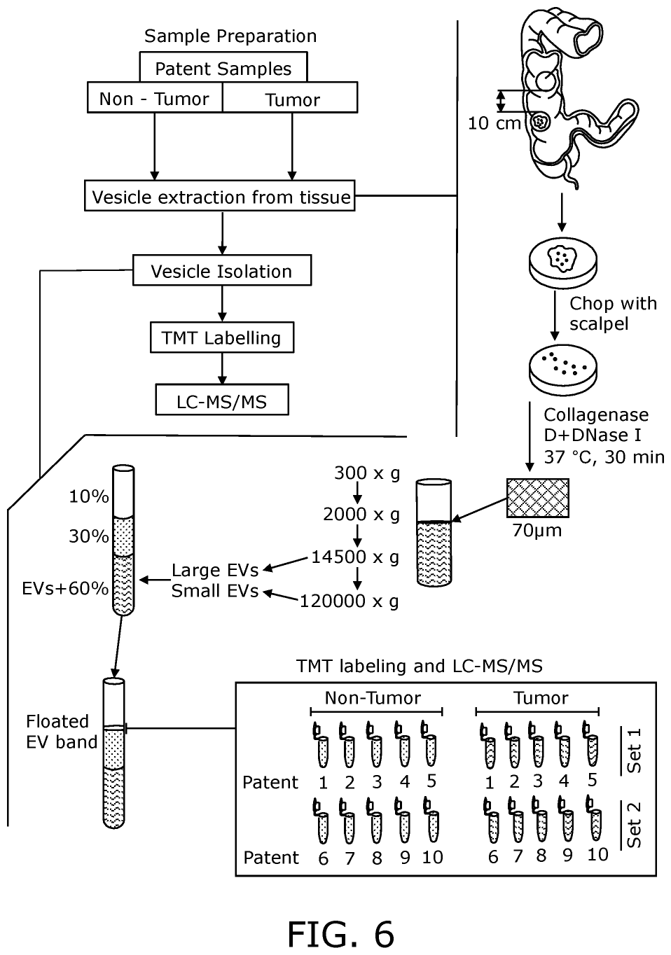

Alternatively, optionally, the method 100 may include ultracentrifugation of the tissue sample to concentrate tissue-resident extracellular vesicles therein, wherein such ultracentrifugation is performed before processing the tissue sample for vesicle extraction. Typically, a combination of both mechanical and enzymatic treatments is used to extract vesicles from the tissue interstitium into an immersion medium, as illustrated and described in FIG. 6. Optionally, the medium, namely conditioned medium after the removal of tissue through filtration and larger colloids and cells by mild centrifugation, is then filtered and carried through a conventional differential ultracentrifugation protocol in order to pellet vesicles followed by purification by floating on an OptiPrep™ density cushion.

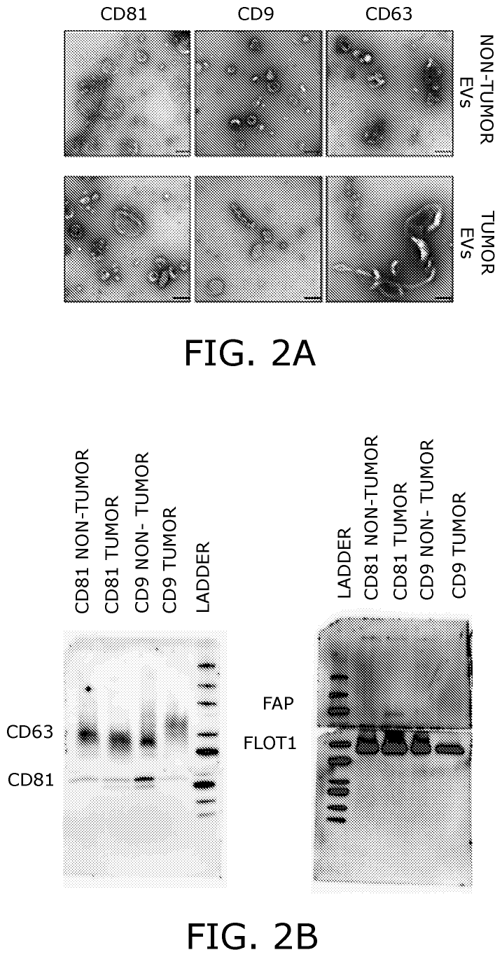

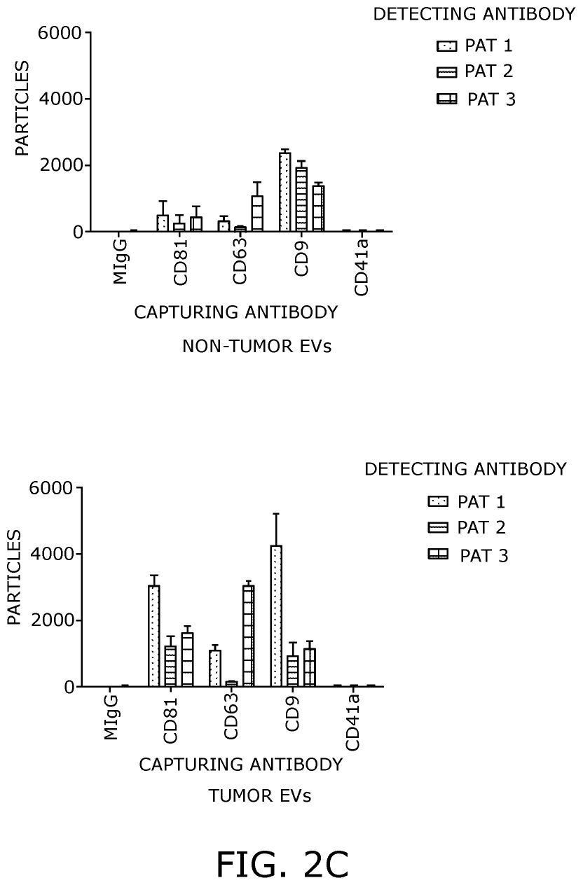

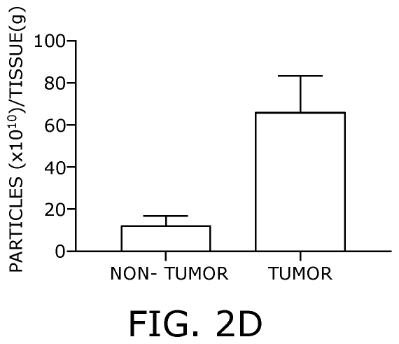

In FIGS. 2A-2C, there is provided an illustration of tissue isolates in a tissue sample, in accordance with an embodiment of the present disclosure. FIG. 2A illustrates electron micrographs showing the vesicle isolates from tumour tissue samples and non-tumour tissue samples of three patients, PAT1, PAT2 and PAT3. As shown, structures characteristic to EVs are illustrated by electron microscopy of both the tumour tissue samples and non-tumour tissue samples. Furthermore, the background seen on the grids appear largely free of scruffy non-vesicular aggregates that may be protein contaminants, thus suggesting successful EV isolation. In FIG. 2B, there are illustrated depictions of tissue isolates in a tissue sample. As shown in FIG. 2B, the Western blots of the tissue sample depict appropriately sized bands of abundant intensities corresponding to membrane proteins CD63, CD81 and Flotillin-1, thereby indicating a high enrichment of tissue-resident EVs in the tissue sample. Notably, the blot appears largely free of scruffy non-vesicular aggregates indicating successful isolation of tissue-resident EVs. As illustrated in FIG. 2C, the bar graph depicts data for particle measurement of the VAPs in tissue samples, in accordance with an embodiment of the present disclosure. The tumour tissue samples of three patients, PAT1, PAT2 and PAT3,comprise increased particle measurement for VAPs, CD81, CD63, CD9 as compared to the non-tumour tissue samples and apparently no change in the CD41 (commonly used as a platelet marker) and the human anti-mouse immunoglobulin (mIgG) (serving as an isotype control) when compared to the non-tumour tissue samples. The Vesicles are captured by immobilized antibodies towards CD9, CD63 and CD81, and subsequently probed with fluorescent antibodies against three epitopes, showing the presence of vesicles double-positive for these markers. As shown in FIG. 2D, the bar graph depicts data for particle measurement of the isolated tissue-resident EVs for two patients, i.e. PAT 1 and PAT 2, from both the tumour tissue samples and non-tumour tissue samples. As shown, an increase of particles (EV load) is observed in tumour tissue samples as compared to the non-tumour tissue samples. ZetaView measurements were performed to investigate the EV load in tissue samples, as shown almost 60 times greater amounts of EVs can be isolated from tumour tissue samples than from non-tumour tissue samples.

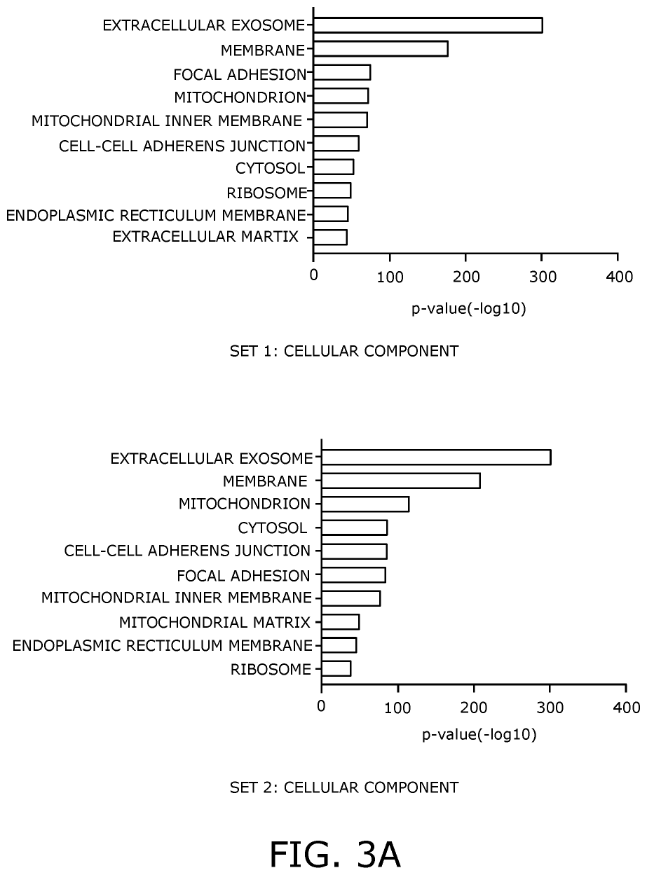

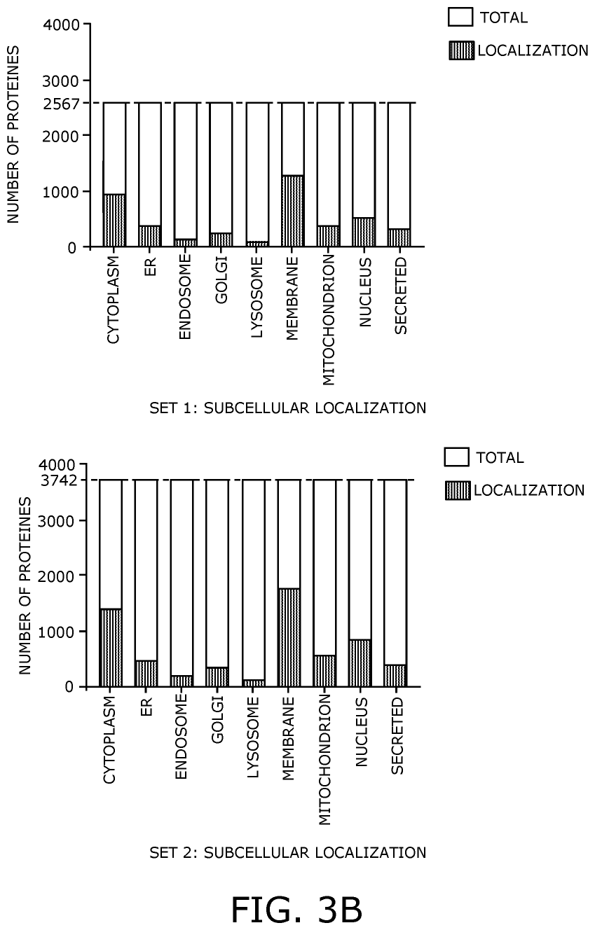

In FIGS. 3A and 3B, there are provided illustrations of various charts to provide a proteomic overview of tissue-derived EVs, in accordance with an embodiment of the present disclosure. In FIG. 3A, there is depicted a Gene Ontology (GO) analysis of the top 10 GO terms for cellular component in terms of p-value associated with the two sets, i.e. Set 1 and Set 2. As shown, GO analysis is capable of demonstrating that “Extracellular Exosome” are the top GO term associated with both the proteomes of Set 1 and Set 2. Also shown are other similar top 10 GO terms, such as “Membrane”, “Focal Adhesion”, “Mitochondrion”, “Mitochondrial Inner Membrane”, “Cell-Cell Adherens Junction”, “Cytosol”, “Ribosome”, “Endoplasmic Reticulum Membrane”, and “Extracellular Matrix” or “Mitochondrial Matrix”. In FIG. 3B, there is depicted the sub-cellular localization of the proteins in the two sets, Set 1 and Set 2. The plain bars show the total amount of VAPs in the two sets and the striped bars show the number of VAPs belonging to a particular cellular localization. Moreover, the two sets show similar traits in terms of gene ontology and subcellular localization of their proteins. This implies that they, despite the difference in numbers, contain a similar distribution of components and thus, to a degree, exemplifies the reproducibility of the procedure. Notably, the protein can belong to several localization groups and thus the total obtained after adding these would exceed the total amount of proteins found.

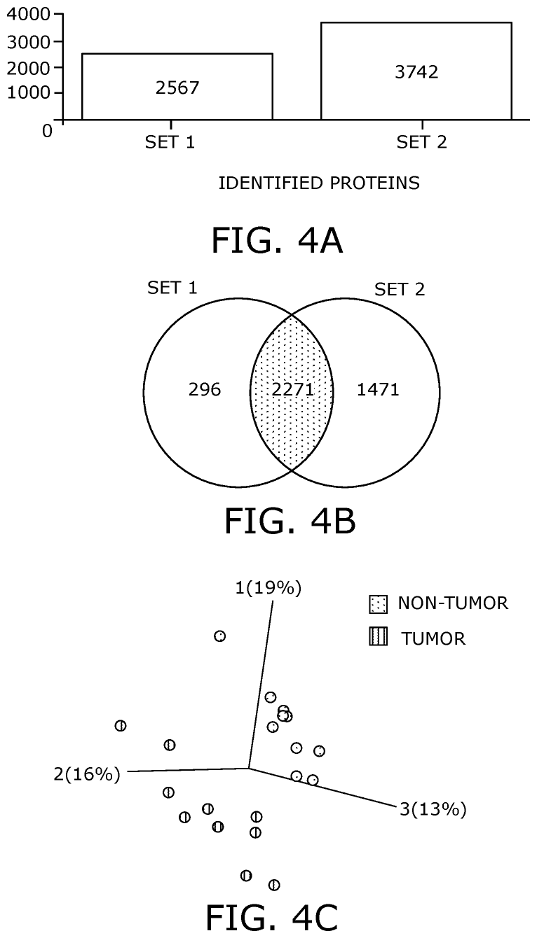

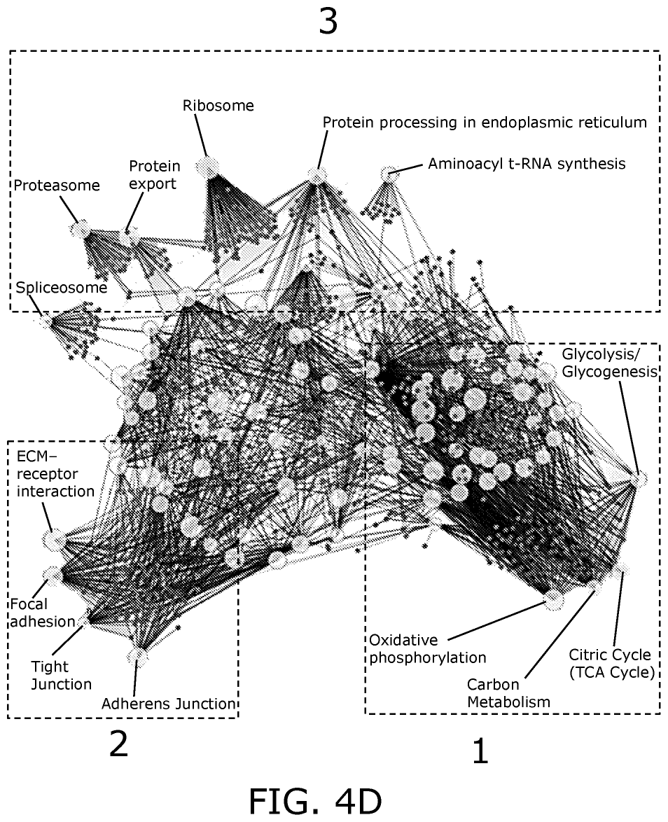

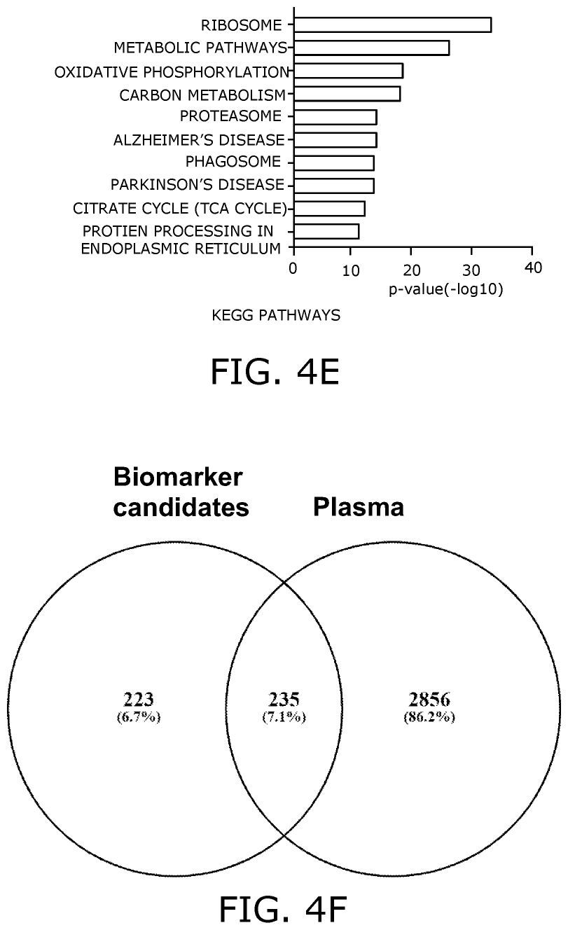

As illustrated in FIGS. 4A to 4E, there is shown a quantitative difference of proteins in tissue-resident EVs derived from the tumour tissue samples, in accordance with an embodiment of the present disclosure. In FIG. 4A, there is provided a depiction of the number of proteins identified in each of the two sets, i.e. Set 1 and Set 2, comprising the tumour-tissue samples and the non-tumour tissue samples collected from ten patients, thereby resulting in a total of 20 samples (10 tumour-tissue samples and 10 non-tumour tissue samples) analysed on two 11-plex TMT sets, obtained by a TMT-labeling approach. Notably, the application of a density gradient during isolation enriches for VAPs and is crucial for proteomic studies of tissue-derived EVs. As shown, the Set 1 comprises 2567 VAPs associated with the tissue-resident EVs and the Set 2 comprises 3742 VAPs associated with the tissue-resident EVs. In FIG. 4B, there is depicted a Venn diagram of Set 1 and Set 2 showing the overlap of the two sets, with 2271 VAPs common between the Set 1 and Set 2. Amongst the two sets, 2271 proteins are found in both the sets. Consequently, since Set 1 contained 2567 proteins in total, most of these are also found in Set 2, while Set 2, although overlapping to a large extent, also has a large portion of unique proteins. Thus, the lower overall overlap of Set 2 can be explained by the shear fact that more proteins are identified in this set. However, over all the two sets are comparable and are therefore beneficially analysed together in the downstream analysis where the proteins identified in both sets were used. In FIG. 4C, there is depicted a principle component analysis (PCA) plot constructed using the overlapping proteins of the Venn diagram of FIG. 4B. The PCA plot demonstrates the relationship among the patient samples. Components ‘1’ (19%), ‘2’ (16%) and ‘3’ (13%) represent 48% of the overall variability in the data. A separation in the three-dimensional plot clearly illustrates that EVs derived from tumour tissue samples and the EVs derived from non-tumour tissue samples form separate clusters and thus differ in composition.

In FIG. 4D, there is shown an illustration of an enrichment analysis network generated using the commonly identified proteins, in accordance with an embodiment of the present disclosure. The enrichment network is constructed in Networkanalyst.ca with the overlapping proteins as input, wherein the nodes represent KEGG pathways. The size of the nodes corresponds to the amounts of genes in that particular pathway that are identified in the input list. As shown, certain nodes containing largely either up- or down-regulated proteins are depicted to visualize more clearly protruding pathways of the network. This results in three larger clusters, of which the bottom two, namely ‘1’ and ‘2’, are skewed towards downregulation in EVs derived from tumour tissue samples as compared to EVs derived from non-tumour tissue samples. These clusters contain proteins are related to either adhesion or energy production. Specifically, amongst the down-regulated clusters in EVs derived from tumour tissue samples are nodes representing “Glycolysis/Glycogenesis” and different aspects of ATP production related to the mitochondria, but also proteins involved in ECM-interaction and junction proteins. The third cluster, i.e. upper one, namely ‘3’, is skewed towards upregulation and related to protein production. In the upregulated cluster, nodes representing protein production and degradation such as “Spliceosome”, “Ribosome” and “Proteasome” amongst others. Taken together, the protrusions of these pathways suggest that the tissue sample to be in a state of low energy production but high protein turnover.

This state is in a cellular context telling of the Warburg effect, in which cancer cells down-regulate oxidative phosphorylation and may favour protein synthesis. Observing this phenotype in vesicles may be a reflection of the cellular state of their origin but could also reflect the fact that these vesicular cargo components are expendable to the cell instead. A general down-regulation of identified proteins related to focal adhesion, tight junctions and extracellular matrix interaction is again interesting if this signature is a reflection of the cell of origin but makes for a conundrum in the vesicular context.

An enrichment analysis network, as provided in FIG. 4D, using Networkanalyst.ca, is generated using the commonly identified VAPs in order to obtain a notion of protruding pathways. Several clusters can be identified where “Metabolic pathways”, being a largely general category, overwhelmingly contains VAPs downregulated in tissue-resident EVs derived from tumour tissue sample. Also, part of this cluster is “Oxidative phosphorylation”, “Carbon metabolism”, “Citric cycle”, “Glycolysis/Glycogenesis” and “Biosynthesis of amino acids”. Also, noteworthy are the “Proteasome” and “Splicosome” clusters which are down-regulated. The up-regulated VAPs are located in several other pathways including “ECM-receptor interaction”, “Focal adhesion” and “Adherent junctions”. By far the most consistently upregulated pathway however is “Ribosome”. Other noteworthy pathways which show a majority of up-regulated VAPs are “Protein export”, “Protein processing in endoplasmic reticulum” and “Endocytosis”.

As illustrated in FIG. 4E, there are shown KEGG pathways of lowest p-values associated with the VAPs present in tumour tissue-derived EVs, in accordance with an embodiment of the present disclosure. As shown, the 10 pathways with lowest p-value as obtained from FIG. 4D provide that the VAPs are associated with these pathways.

FIG. 4F depicts the number of biomarker candidates identified in plasma, in accordance with an embodiment of the present disclosure. As shown, the plasma comprises 2856 proteins, out of which 235, i.e. 7.1%, of the total proteins are suitable as biomarker candidates.

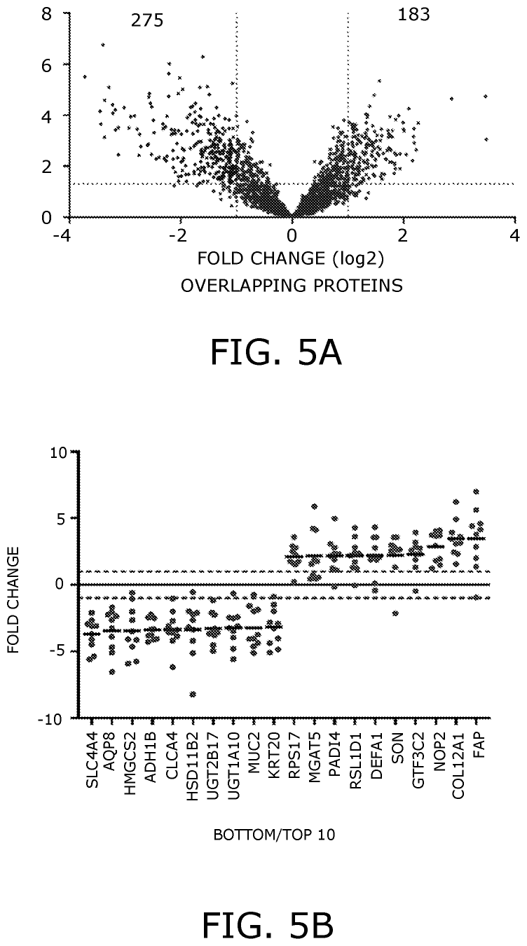

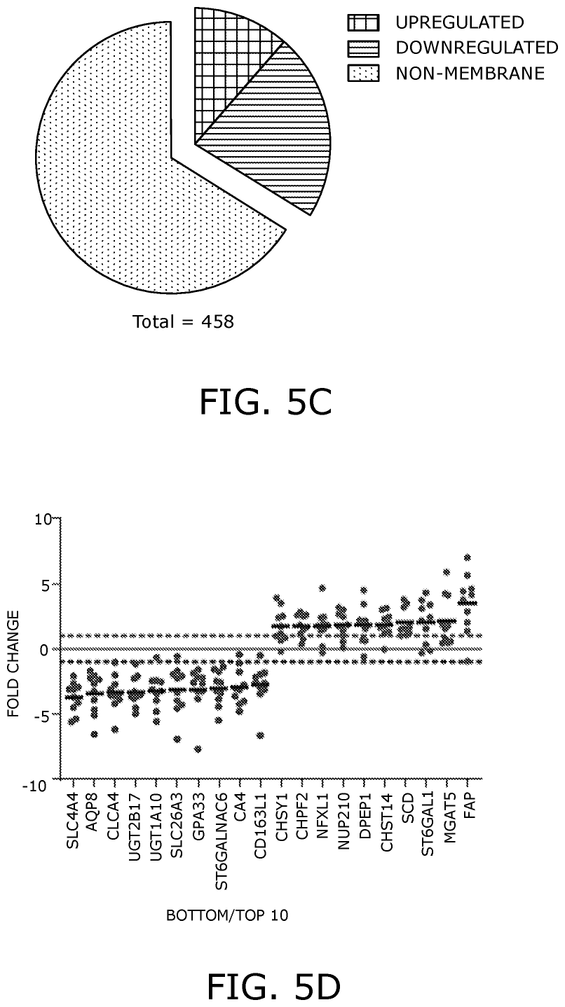

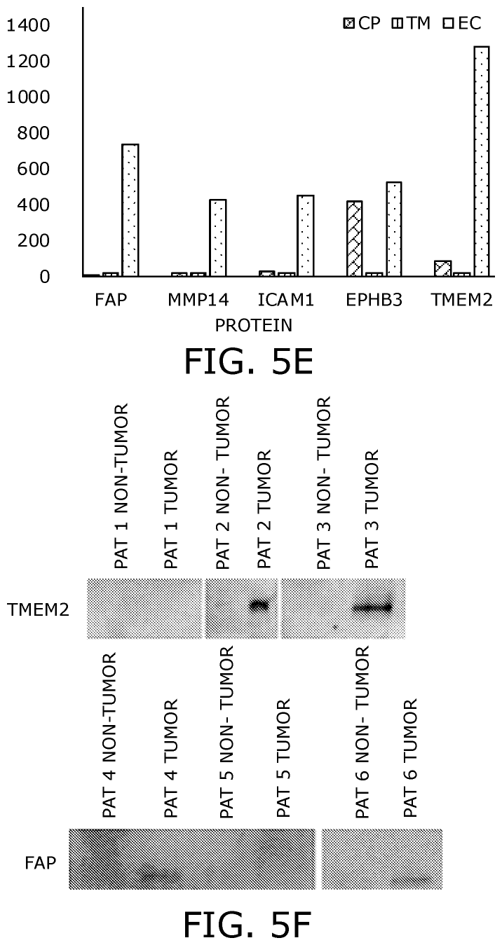

As illustrated in FIGS. 5A-5D, there is shown a quantitative difference of up-regulated and down-regulated proteins in tissue-resident EVs derived from the tumour tissue samples and non-tumour tissue samples, in accordance with an embodiment of the present disclosure. FIG. 5A depicts a Volcano plot of the VAPs common for both the sets. The dotted lines indicate the cut off, which is 1.3 on the Y-axis (corresponding to a p-value of 0.05) and 1 on the X-axis (corresponding to a fold change of 2). A positive fold change (right of 1.3) is representative of an enrichment in tissue-resident EVs derived from tumour tissue sample, while a negative fold change (left of −1.3) represents a depletion in tissue-resident EVs derived from tumour tissue sample. As shown, a total of 183 VAPs are up-regulated in tissue-resident EVs derived from the tumour tissue samples and 275 downregulated. FIG. 5B represents the top 10 up-regulated and top 10 down-regulated proteins in each set according to fold change. The positive fold change (above 0 on the Y-axis) is representative of up-regulated proteins, while a negative fold change (below 0 on the Y-axis) is representative of down-regulated proteins. For example, overexpression of prolyl endopeptidase Fibroblast activating factor FAP (FAP), which can be both a membrane bound protein or shed, has been linked to negative prognosis in colon cancer. Similarly, Collagen alpha-1 (XII) chain (COL12A1), has been suggested to be associated with poor prognosis. Some of the most downregulated proteins in tissue-resident EVs derived from tumour tissue sample could point to a de-differentiation of epithelial cells through the loss of epithelia-typical proteins, such as transporters amongst others. To exemplify, down-regulation of the chloride/bicarbonate exchanger SLC6A3 has been shown to be coupled to the de-differentiation of colon epithelia in cancer, as seems to be the case for MUC2. Again, this further fortifies a confidence in the isolates as being a product of the disease phenotype. As such, the vesicular cargo would be of great value as biomarkers of disease if they can be detected in circulation. As shown in FIG. 5B, proteins such as prolyl endopeptidate FAP (FAP) and electrogenic sodium bicarbonate cotransporter 1 (SLC4A4) are dysregulated in tissue-resident EVs derived from the tumour tissue samples.

FIG. 5C represents a pie chart showing the significantly up- and down-regulated EV-associated or membrane proteins. The membrane subcellular localizations acquired from Uniprot IDs which reveals 52 up-regulated and 103 down-regulated VAPs amongst the proteins with a p-value of 0.05 and/or a fold change of 2. FIG. 5D represents the top 10 up-regulated VAPs and top 10 down-regulated VAPs, i.e. membrane proteins, displayed on vesicular surfaces in each set. The positive fold change (above 0 on the Y-axis) is representative of up-regulated VAPs, while a negative fold change (below 0 on the Y-axis) is representative of down-regulated VAPs. As shown in FIG. 5D, VAPs such as prolyl endopeptidate FAP (FAP) and electrogenic sodium bicarbonate cotransporter 1 (SLC4A4) are dysregulated in tissue-resident EVs derived from the tumour tissue samples. The membrane association of proteins, such as FAP and SLC4A4, further strengthens a claim that they are indeed vesicle-bound elements and their particular position on the vesicles make them attractive from a biomarker perspective as targets for affinity-based isolation from biofluids.

As illustrated in FIG. 5E, there is shown a bar graph depicting topology of the membrane proteins, in accordance with an embodiment of the present disclosure. As shown, membrane topology, in cytoplasm, membrane and extracellular vesicle of 5 hand-picked proteins, i.e. FAP, MMP14, ICAM1, EPHB3 and TMEM2, is provided. Five proteins appeared to be especially suited for such an approach, owing to their membrane topology where a decent portion is extracellularly displayed. Shown are the gene names, the number of amino acids resident in the cytoplasm (CP), transmembrane (TM), and extracellularly (EC). As shown, the proteins are found in abundance in the tissue-resident EVs, therefore are potential biomarkers for colon-cancer. Additionally, three of the five proteins, namely FAP, MMP14 and TMEM2 are enzymes capable of degrading extracellular matrix, which apart from the potential to facilitate the escape of the cell from its tissue microenvironment could also facilitate the vesicular escape. It will be appreciated that to increase the suitability as targets for pull down assays, up-regulated transmembrane proteins which localize to the plasma membrane with substantial epitopes presented on the extracellular face of the vesicles are hand-picked. This approach dramatically reduces the list to only a handful of proteins as listed in FIG. 5E.

As shown in FIG. 5F, the Western blots of the VAPs, FAP and TMEM2, depict appropriately sized bands of abundant intensities corresponding to membrane proteins FAP and TMEM2 as measured by mass spectrometry, thereby indicating a high enrichment of tissue-resident EVs in the tissue sample.

As illustrated in FIG. 6, there is shown a flow chart depicting of steps of a method 600 for sample preparation for identifying colon cancer-specific vesicle-associated proteins therefrom, in accordance with an embodiment of the present disclosure. The method comprises, at a step 602, obtaining a tissue sample, namely a tumour tissue sample and a non-tumour tissue sample, from a patient; at a step 604, extracting tissue-resident extracellular vesicles from the tissue sample; at a step 606, isolating vesicle-associated proteins associated with the isolated tissue-resident extracellular vesicles; at a step 608, quantifying the identified vesicle-associated proteins by TMT labeling; and at a step 610, analysing vesicle-associated proteins.

As shown, tissue samples are collected from colorectal cancer patients during surgery, wherein each patient yields one tumour tissue sample and one non-tumour tissue sample to serve as control. The non-tumour tissue sample is collected from a site 10 cm away from the tumour tissue sample. The tissue samples, immediately after excision, are subjected to vesicle extraction. The extraction is performed by a combination of both mechanical and enzymatic techniques. By cutting the tissue into smaller pieces, optionally by using a scalpel or a chopper of suitable features, such as sharpness, sterility, and so forth, vesicles are allowed to more readily diffuse into the RPMI medium, comprising Collagenase D and DNase 1 at 37° C. for 30 minutes, for digestion of tissue sample. Notably, Collagenase D digestion further degrades the extracellular matrix while DNase 1 prevents the formation of a sticky pellet further downstream in the isolation protocol. Following the release of vesicles into the RPMI medium, filtration and a sequential centrifugation protocol is applied to eliminate large tissue pieces, free cells and cell debris. A 70 μm filter is used for filtering tissue digested samples. Lastly, EVs are isolated through an ultracentrifugation protocol with a density cushion floatation as the last step, where buoyant vesicles can be collected at the intersection of 1.078 g/mL and 1.175 g/mL Optiprep. Protein extraction, digestion and TMT-labeling precedes the final step of mass spectrometric analysis.

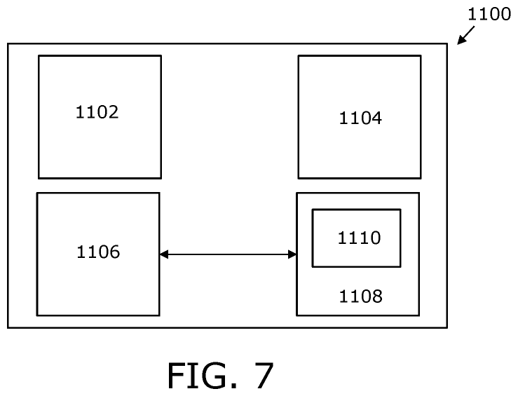

As illustrated in FIG. 7, there is shown a block diagram of a system 700 for identifying colon cancer-specific vesicle-associated proteins from a tissue sample of a subject, in accordance with an embodiment of the present disclosure. The system 700 includes: (a) a kit 702 for isolating tissue-resident extracellular vesicles from the tissue sample; (b) a mass spectrometer 704 for identifying vesicle-associated proteins associated with the isolated tissue-resident extracellular vesicles; (c) a database 706 having stored therein pre-determined vesicle-associated protein profiles of the tumour tissue samples and/or non-tumour tissue samples; and (d) a computing unit 708 in communication with the database 706. The computing unit 708 includes memory 710 in which is stored executable codes operable to:
(i) quantify the identified vesicle-associated proteins to identify one or more major vesicle-associated proteins,
(ii) create vesicle-associated protein profiles for the identified vesicle-associated proteins, and
(iii) compare the vesicle-associated protein profiles for the identified vesicle-associated proteins with the pre-determined vesicle-associated protein profiles of the tumour tissue samples and/or non-tumour tissue samples.

Typically, the system 700 includes an isolation arrangement, such as the kit 702, and an identifying arrangement, such as the mass spectrometer 704. The kit 702 is operable, namely configured, to isolate the tissue-resident EVs from the tissue sample of the subject and is communicably coupled to the mass spectrometer 704. The mass spectrometer 704 may be operable, namely configured, to identify the VAPs associated with the isolated tissue-resident EVs. The mass spectrometer 704 is operable, namely configured, to recognize one or more VAPₘₐⱼ from the identified VAPs. The computing unit 706 is operable, namely configured, to quantify the identified VAPs to identify one or more VAPₘₐⱼ. The computing unit 706 is further operable to create VAP profiles for the identified one or more VAPₘₐⱼ and compare the VAPₘₐⱼ profiles of the identified one or more VAPₘₐⱼ with the pre-determined VAP profiles of the tumour tissue samples and/or non-tumour tissue samples.

Optionally, the mass spectrometer 704 may be used for separating the VAPs and the one or more VAPₘₐⱼ. In an example, the computing unit 1106 may be operable, namely configured, to employ a Protein Assay for quantifying the identified VAPs to identify one or more VAPₘₐⱼ.

Optionally, the kit 702 comprises a laboratory equipment for isolating the tissue-resident extracellular vesicles from a tissue sample; wherein the laboratory equipment comprises any of a surgical arrangement, an RT-PCR, test tubes, pipettes, assay plates, a centrifuge, a 70-micrometer filter, a density gradient, a quantification system, an electron microscope, an analyser, a mass spectrometer. Furthermore, the kit 702 comprises one or more reagents selected from a group consisting of: RPMI medium, DNase 1®, Collagenase D®, PBS medium, OptiPrep™ SDS, digestion buffer (trypsin/sodium deoxycholate), dithiothreitol, urea, TMT 11-plex isobaric mass tagging reagents®, TFA, ammonium formate buffer (formic acid), acetonitrile. Furthermore, the kit 702 comprises an epitope-specific binder against colon cancer-associated vesicle-associated proteins, and at least one colon cancer-associated marker detection agent. More optionally, the laboratory equipment comprises a surgical arrangement, such as a knife, a scalpel, a blade, a pair of surgical scissors, and so forth, for isolating the tissue-resident extracellular vesicles from the tissue sample. The laboratory equipment may further comprise at least one slicer for slicing the tissue sample into fragments, at least one incubator for incubating the fragments with one or more enzymes to release the extracellular vesicles, and at least one centrifuge for segregating tissue debris and the extracellular vesicles from the incubated fragments. In an example, incubation of the fragments of the tissues may be performed in the presence of one or more enzymes selected from a group of proteases including a matrix metalloproteinase, collagenases, and papain and nucleases including DNase, RNase, and Benzonase. Optionally, the kit 702 may include a filtration arrangement, such as a 70 μm filter, for separating tissue debris and the extracellular vesicles from centrifuged suspension of the incubated fragments. Optionally, the epitope-specific binder binds to colon cancer-associated VAPs and enable detecting a VAP associated with VAPs. Optionally, the kit 72 comprises at least one colon cancer-associated marker detection agent for detecting the said colon cancer-associated VAPs.

Optionally, the computing unit 708 is further operable to obtain intensity information of the identified vesicle-associated proteins for quantification, by employing a labelling tool. More optionally, the computing unit 708 employs a label-free quantification tool or an isotope labeling-based quantification tool to obtain intensity information of the identified VAPs or one or more VAPₘₐⱼ Further, the computing unit 708 is operable to compare expressional differences in VAPs associated with the tissue-resident EVs derived from tumour tissue sample from VAPs associated with the tissue-resident EVs derived from non-tumour tissue sample. For example, the computing unit 708 may perform a search on a basis of at least one parameter including enzyme specificity, a variable modification, a fixed modification, oxidation properties, carbamidomethylation properties and an ion tolerance. Furthermore, the intensity information of the identified VAPs or one or more VAPₘₐⱼ may include, but is not limited to, the concentration and/or sequence lengths of the one or more major vesicle-associated proteins.

In an embodiment, the computing unit 708 may be operable, namely configured, to employ a nanoFCM analysis, ELISA, alphaLISA, FACS, fluorescent correlation microscopy, immune-electron microscopy, or other methods, for comparing expressional differences in VAPs associated with the tissue-resident EVs derived from tumour tissue sample against VAPs associated with the tissue-resident EVs derived from non-tumour tissue sample.

Optionally, the system 700 may be operable, namely configured, to create pre-determined VAP profiles of the tumour tissue samples and/or non-tumour tissue samples. For example, the kit 702 may be operable to isolate tissue-resident EVs from the tumour tissue samples and/or non-tumour tissue samples, and the mass spectrometer 704 may be operable, namely configured, to identify the VAPs associated with the isolated tissue-resident EVs. Furthermore, the computing unit 708 may be operable, namely configured, to quantify the identified VAPs by employing the Protein Assay.

Optionally, the computing unit 708 is operationally connected to a Label-free quantification tool and a Protein Sequences database via at least one communication network (not shown). Furthermore, the Protein Sequences database may include, but is not limited to, MaxQuant® quantification tool with an Andromeda® search engine.

It will be appreciated that the computing unit 708 may include an inbuilt database associated with the Label-free quantification tool for obtaining intensity information relating to the one or more VAPₘₐⱼ and a Protein Sequences database for searching the identified VAPs. However, in most examples, the Label-free quantification tool and a Protein Sequences database of the system 700 are external server-based arrangements.

The present disclosure further provides a computer program product including non-transitory computer-readable storage medium having computer-readable instructions stored thereon, the computer-readable instructions being executable by a computerized device comprising processing hardware to execute the method 100 for identifying colon cancer-specific vesicle-associated proteins from a tissue sample of a subject, shown and described with FIG. 1 and/or FIG. 6.

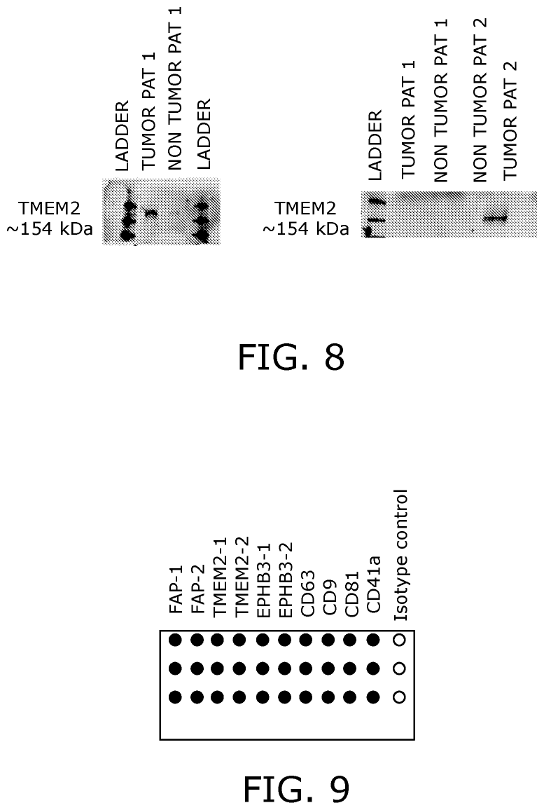

As illustrated in FIG. 8, there is shown Western blots for TMEM2, in accordance with an embodiment of the present disclosure. The tumour tissue samples from two patients, i.e. PAT 1 (left) and PAT1 (right) and PAT 2 (right), depict appropriately sized bands of abundant intensities corresponding to the membrane proteins TMEM2, when compared to cancer-specific proteins as observed in A549 (a human lung carcinoma) cell lysate, used as a control. However, the non-tumour tissue samples from the two patients did not show any bands corresponding to TMEM2. As evident, the VAP, TMEM2, may be a potential biomarker for colon-cancer. It will be appreciated that PAT1 (left) and PAT1 (right) for both tumour and non-tumour tissue samples may be from different patient without any limitations.

As illustrated in FIG. 9, there is depicted a proposed method to screen samples for the potential biomarkers, in accordance with an embodiment of the present disclosure. As shown, a chip is spotted with antibodies against FAP, TMEM2, and EPHB3. The antibodies against FAP, TMEM2, and EPHB3 are shown in triplicates (columns). Moreover, each of these three markers are spotted with two different clones, i.e. FAP-1, FAP-2, TMEM2-1, TMEM2-2, EPHB3-1 and EPHB3-2.

Furthermore, CD63, CD9 and CD81 are commonly used EV markers and do not form part of the discovery but are useful nonetheless. CD41a is a platelet marker and thus is useful as a “contamination” control when dealing with certain samples. Isotype control is for detecting background/unspecific binding.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

Experimental Details

Transmission Electron microscopy: VAPs analysis was achieved by negative staining method. Notably, negative staining is often used in diagnostic microscopy, such as transmission electron microscopy, for contrasting a thin specimen with an optically-opaque fluid staining as a background. Commonly used negative stains that scatter electrons strongly include ammonium molybdate, auroglucothionate, uranyl acetate, uranyl formate, osmium tetroxide and osmium ferricyanide and phosphotungstic acid. Briefly, 10 μg of tissue sample is adsorbed to a glow-discharged carbon-coated copper (200-mesh) grid (Electron Microscopy Sciences, Hatfield Pa., US). After two washes with deionized water, tissue-resident EVs are fixed in 2.5% glutaraldehyde. After further two washes with deionized water, the tissue samples are contrasted with 2% uranyl acetate for 1.5 min. Electron micrograms are obtained using a digitized LEO 912AB Omega Transmission Electron Microscope (Carl Zeiss SMT AG, Mainz, Germany) at 120 kV equipped with a Veleta CCD camera (Olympus-SiS, Münster, Germany). It will be appreciated that the tissue samples are beneficially imaged at room temperature.

Nanoparticle tracking analysis (NTA): Nanoparticle tracking analysis is an established method for visualizing and analysing nanoparticle size distribution in a liquid suspension, by utilizing the properties of both light scattering and Brownian motion. Particle concentration is determined for both tumour tissue samples and non-tumour tissue samples from three patients. Briefly, an aliquot of the purified vesicle-associated proteins, isolated after sequential centrifugation, is diluted to 1000-10000 times in PBS and loaded into a sample chamber. The sample chamber is illuminated by a light source, such as a laser beam. The particles in the path of the beam scattered the laser light which is measured using a ZetaView® PMX 110 instrument (Particle Metrix) and data is beneficially analyzed using the ZetaView® analysis software version 8.2.30.1 with a minimum size of 5, a maximum size of 5000, and a minimum brightness of 20.

ExoView™ analysis: Surface epitopes of vesicles are susceptible to being evaluated using an ExoView™ Plasma Tetraspanin kit and an ExoView™ R100 (NanoView Biosciences, Boston, Mass., US), according to manufacturer's instructions. ExoView™ Plasma Tetraspanin kit provides multi-level and comprehensive characterization of EVs for particle size analysis, EVs count, EVs phenotype, EV cargo, and biomarker co-localization. The ExoView™ Plasma Tetraspanin kit system (NanoView Biosciences) is a chip-based microscopy system utilizing: (a) surface-immobilized antibodies for capturing EVs, and (b) fluorescently-labeled antibodies for biomarker detection and co-localization as well as light-scattering for particle size analysis. Briefly, chips coated with antibodies toward CD81, CD63 and CD9 are beneficially pre-scanned to measure non-sample particles that might be pre-existing on the chip. The concentration of EVs is measured with NTA and the tissue samples of desired concentration (1×10⁸ particles) are then diluted to a suitable dilution (1:1) using an Incubation Solution and loaded onto the chips which are incubated at room temperature overnight. Briefly, for example, thirty-five microliter samples are susceptible to being loaded onto the chips and the chips are subsequently washed three times with the Incubation Buffer followed by incubation of fluorescently-labeled antibodies targeting CD81, CD63 and CD9 diluted in the Incubation Buffer supplemented with 1% BSA for one hour at room temperature. Chips are further washed with a Washing Buffer three times followed by washing with a Rinse Buffer after which they are scanned using the ExoView™ system. Moreover, analysis is beneficially performed using the NanoViewer™ software running on version 2.6.0 (NanoView Biosciences).

NanoFCM analysis: The nanoFCM analysis includes detecting single particles similar to FACS. In particular, the tissue-resident EVs derived from the tumour tissue samples are incubated with either of PE-conjugated CD81 or CD47 antibodies for 30 minutes at 37° C. followed by recovering the tissue-resident EVs by ultracentrifugation at 100,000×g_{avg} (Type120.1) for 15 minutes and recovered tissue-resident EVs are re-suspended with PBS. The stained tissue-resident EV's are subjected to nanoFCM analysis for identifying PE signals for single tissue-resident EV. Thereafter, tissue-resident EVs derived from non-tumour samples are used as control for overlapping the PE signals for various EVs and detecting biomarker in VAPs of the tissue-resident EVs of the subject.

Enrichment analysis: The web-based tool NetworkAnalyst (https://www.networkanalyst.ca, Jan. 12, 2020) is beneficially used to generate a network based on the 2271 proteins identified in both sets by drawing data from the KEGG database (https://www.genome.jp/kegg/).

Correlative up-regulation of VAPs: To generate a list of high confidence VAPs, a cut off is beneficially set. Apart from being identified in both sets, a VAP would need a fold change of at least 2 and a p-value of 0.05 or lower to be confidently considered as differently loaded in the tissue-resident EVs derived from the tumour tissue sample as compared to the tissue-resident EVs derived from the non-tumour tissue sample. As such, 183 proteins are beneficially considered to be up-regulated in tissue-resident EVs derived from tumour tissue sample and 275 down-regulated. The top 10 up- and down-regulated proteins and their individual patient fold changes are thereby obtained. Highly expressed components could be potential biomarker candidates and a panel of such components could potentially be of greater value than a single marker. Thus, correlations are beneficially calculated in order to tease out if the dataset contained any VAPs with similar expression trends. For example, three groups of VAPs showed good correlation amongst VAPs where FN1, COL12A and FAP formed a group. Another group was formed by DEFA1 and PADI4 while a third group consisted of CHPF2 and CHST14.

Principal Component Analysis: A principal component analysis (PCA) is beneficially performed using the Qlucore Omics Explorer software (Qlucore, Lund, Sweden). The three components that best explain the variability in the data are plotted for the 2271 overlapping VAPs identified in the two sets, Set 1 and Set 2.

Statistics and calculations: To calculate the significance between VAP quantities of the tissue-resident EVs derived from the non-tumour tissue samples and the tissue-resident EVs derived from the non-tumour tissue samples, Student T-tests are beneficially performed on logged values. A p-value of 0.05 or lower is considered as being significant. Fold changes are calculated between each paired tumour tissue sample and non-tumour tissue sample. Negative values are generated by division, using ‘−1’ as numerator and the calculated value (if below ‘1’) as the denominator.

Appendix to the Specification

TABLE 3

| Protein | Uniprot | |
|---|---|---|
| name | ID | Description |
| FAP | Q12884 | Prolyl endopeptidase FAP OS = Homo sapiens GN = FAP PE = 1 |
| | | SV = 5 |
| MGAT5 | Q09328 | Alpha-1,6-mannosylglycoprotein 6-beta-N- |
| | | acetylglucosaminyltransferase A OS = Homo sapiens |
| | | GN = MGAT5 PE = 2 SV = 1 |
| ST6GAL1 | P15907 | Beta-galactoside alpha-2,6-sialyltransferase 1 OS = Homo |
| | | sapiens GN = ST6GAL1 PE = 1 SV = 1 |
| SCD | O00767 | Acyl-CoA desaturase OS = Homo sapiens GN = SCD PE = 1 SV = 2 |
| CHST14 | Q8NCH0 | Carbohydrate sulfotransferase 14 OS = Homo sapiens |
| | | GN = CHST14 PE = 1 SV = 2 |
| DPEP1 | P16444 | Dipeptidase 1 OS = Homo sapiens GN = DPEP1 PE = 1 SV = 3 |
| NUP210 | Q8TEM1 | Nuclear pore membrane glycoprotein 210 OS = Homo sapiens |
| | | GN = NUP210 PE = 1 SV = 3 |
| NFXL1 | Q6ZNB6 | NF-X1-type zinc finger protein NFXL1 OS = Homo sapiens |
| | | GN = NFXL1 PE = 1 SV = 2 |
| CHPF2 | Q9P2E5 | Chondroitin sulfate glucuronyltransferase OS = Homo sapiens |
| | | GN = CHPF2 PE = 2 SV = 2 |
| CHSY1 | Q86X52 | Chondroitin sulfate synthase 1 OS = Homo sapiens GN = CHSY1 |
| | | PE = 1 SV = 3 |
| FUT6 | P51993 | Alpha-(1,3)-fucosyltransferase 6 OS = Homo sapiens GN = FUT6 |
| | | PE = 1 SV = 1 |
| CERS6 | Q6ZMG9 | Ceramide synthase 6 OS = Homo sapiens GN = CERS6 PE = 1 |
| | | SV = 1 |
| GALNT6 | Q8NCL4 | Polypeptide N-acetylgalactosaminyltransferase 6 OS = Homo |
| | | sapiens GN = GALNT6 PE = 2 SV = 2 |
| MMP14 | P50281 | Matrix metalloproteinase-14 OS = Homo sapiens GN = MMP14 |
| | | PE = 1 SV = 3 |
| AGRN | O00468 | Agrin OS = Homo sapiens GN = AGRN PE = 1 SV = 5 |
| ICAM1 | P05362 | Intercellular adhesion molecule 1 OS = Homo sapiens |
| | | GN = ICAM1 PE = 1 SV = 2 |
| SEL1L3 | Q68CR1 | Protein sel-1 homolog 3 OS = Homo sapiens GN = SEL1L3 PE = 1 |
| | | SV = 2 |
| FAT1 | Q14517 | Protocadherin Fat 1 OS = Homo sapiens GN = FAT1 PE = 1 SV = 2 |
| FCGR1A | P12314 | High affinity immunoglobulin gamma Fc receptor I OS = Homo |
| | | sapiens GN = FCGR1A PE = 1 SV = 2 |
| FKBP11 | Q9NYL4 | Peptidyl-prolyl cis-trans isomerase FKBP11 OS = Homo sapiens |
| | | GN = FKBP11 PE = 1 SV = 1 |
| DDX46 | Q7L014 | Probable ATP-dependent RNA helicase DDX46 OS = Homo |
| | | sapiens GN = DDX46 PE = 1 SV = 2 |
| GBP1 | P32455 | Guanylate-binding protein 1 OS = Homo sapiens GN = GBP1 PE = 1 |
| | | SV = 2 |
| TMCO1 | Q9UM00 | Calcium load-activated calcium channel OS = Homo sapiens |
| | | GN = TMCO1 PE = 1 SV = 1 |
| EPHB3 | P54753 | Ephrin type-B receptor 3 OS = Homo sapiens GN = EPHB3 PE = 1 |
| | | SV = 2 |
| MME | P08473 | Neprilysin OS = Homo sapiens GN = MME PE = 1 SV = 2 |
| NDC1 | Q9BTX1 | Nucleoporin NDC1 OS = Homo sapiens GN = NDC1 PE = 1 SV = 2 |
| TMEM2 | Q9UHN6 | Cell surface hyaluronidase OS = Homo sapiens GN = TMEM2 PE = 1 |
| | | SV = 1 |
| LILRB3 | O75022 | Leukocyte immunoglobulin-like receptor subfamily B member 3 |
| | | OS = Homo sapiens GN = LILRB3 PE = 1 SV = 3 |
| CYP4F3 | Q08477 | Docosahexaenoic acid omega-hydroxylase CYP4F3 OS = Homo |
| | | sapiens GN = CYP4F3 PE = 1 SV = 2 |
| STT3B | Q8TCJ2 | Dolichyl-diphosphooligosaccharide--protein glycosyltransferase |
| | | subunit STT3B OS = Homo sapiens GN = STT3B PE = 1 SV = 1 |
| DNAJA1 | P31689 | DnaJ homolog subfamily A member 1 OS = Homo sapiens |
| | | GN = DNAJA1 PE = 1 SV = 2 |
| TMEM214 | Q6NUQ4 | Transmembrane protein 214 OS = Homo sapiens GN = TMEM214 |
| | | PE = 1 SV = 2 |
| TMEM63A | O94886 | CSC1-like protein 1 OS = Homo sapiens GN = TMEM63A PE = 1 |
| | | SV = 3 |
| FUT8 | Q9BYC5 | Alpha-(1,6)-fucosyltransferase OS = Homo sapiens GN = FUT8 |
| | | PE = 1 SV = 2 |
| TAPBP | O15533 | Tapasin OS = Homo sapiens GN = TAPBP PE = 1 SV = 1 |
| EXT2 | Q93063 | Exostosin-2 OS = Homo sapiens GN = EXT2 PE = 1 SV = 1 |
| CERS2 | Q96G23 | Ceramide synthase 2 OS = Homo sapiens GN = CERS2 PE = 1 |
| | | SV = 1 |
| GGCX | P38435 | Vitamin K-dependent gamma-carboxylase OS = Homo sapiens |
| | | GN = GGCX PE = 1 SV = 2 |
| CEACAM5 | P06731 | Carcinoembryonic antigen-related cell adhesion molecule 5 |
| | | OS = Homo sapiens GN = CEACAM5 PE = 1 SV = 3 |
| RPL15 | P61313 | 60S ribosomal protein L15 OS = Homo sapiens GN = RPL15 PE = 1 |
| | | SV = 2 |
| FUT4 | P22083 | Alpha-(1,3)-fucosyltransferase 4 OS = Homo sapiens GN = FUT4 |
| | | PE = 2 SV = 3 |
| GLCE | O94923 | D-glucuronyl C5-epimerase OS = Homo sapiens GN = GLCE PE = 1 |
| | | SV = 3 |
| MAN2A2 | P49641 | Alpha-mannosidase 2× OS = Homo sapiens GN = MAN2A2 PE = 2 |
| | | SV = 3 |
| RHBDF2 | Q6PJF5 | Inactive rhomboid protein 2 OS = Homo sapiens GN = RHBDF2 |
| | | PE = 1 SV = 2 |
| TMX2 | Q9Y320 | Thioredoxin-related transmembrane protein 2 OS = Homo |
| | | sapiens GN = TMX2 PE = 1 SV = 1 |
| ENTPD6 | O75354 | Ectonucleoside triphosphate diphosphohydrolase 6 OS = Homo |
| | | sapiens GN = ENTPD6 PE = 1 SV = 3 |
| FAM57A | Q8TBR7 | Protein FAM57A OS = Homo sapiens GN = FAM57A PE = 1 SV = 2 |
| CHMP3 | Q9Y3E7 | Charged multivesicular body protein 3 OS = Homo sapiens |
| | | GN = CHMP3 PE = 1 SV = 3 |
| HM13 | Q8TCT9 | Minor histocompatibility antigen H13 OS = Homo sapiens |
| | | GN = HM13 PE = 1 SV = 1 |
| GPAA1 | O43292 | Glycosylphosphatidylinositol anchor attachment 1 protein |
| | | OS = Homo sapiens GN = GPAA1 PE = 1 SV = 3 |
| SEC62 | Q99442 | Translocation protein SEC62 OS = Homo sapiens GN = SEC62 |
| | | PE = 1 SV = 1 |
| IKBIP | Q70UQ0 | Inhibitor of nuclear factor kappa-B kinase-interacting protein |
| | | OS = Homo sapiens GN = IKBIP PE = 1 SV = 1 |

1.-15. (canceled)

16. A method of identifying colon cancer-specific vesicle-associated proteins from a tissue sample of a subject, characterized in that the method comprises steps of:
(a) isolating tissue-resident extracellular vesicles from the tissue sample;
(b) identifying vesicle-associated proteins associated with the isolated tissue-resident extracellular vesicles;
(c) quantifying the identified vesicle-associated proteins to identify one or more major vesicle-associated proteins;
(d) creating vesicle-associated protein profiles for the identified vesicle-associated proteins; and
(e) comparing the vesicle-associated protein profiles for the identified vesicle-associated proteins with pre-determined vesicle-associated protein profiles of the tumour tissue samples and/or non-tumour tissue samples.

17. A method of claim 16, characterized in that, at the step (a) of isolating tissue-resident extracellular vesicles, the method includes:
(i) processing the tissue sample to obtain a tissue conditioned fluid;
(ii) collecting the tissue-resident extracellular vesicles from the tissue conditioned fluid; and
(iii) purifying the collected tissue-resident extracellular vesicles by a density gradient preparation.

18. A method of claim 17, characterized in that, at the step (i) of processing the tissue sample, the method includes:
slicing the tissue sample into fragments;
incubating the fragments with one or more reagents in an assay plate under controlled conditions; and
separating the tissue-resident extracellular vesicles from the tissue debris to obtain a tissue conditioned fluid.

19. A method of claim 18, characterized in that the controlled conditions include an agitation in a range of 2 to 500 rotations per minute at a temperature of 37° C. for a time period of 30 minutes, and filtering through a 70-micrometre filter.

20. A method of claim 18, characterized in that the one or more reagents are selected from a group of growth medium including a RPMI medium, proteases including a matrix metalloproteinase, collagenases including a collagenase D, and papain and nucleases including DNase 1, RNase, and Benzonase.

21. A method of claim 18, characterized in that each of the fragments weigh in a range of 0.01 to 0.25 milligram.

22. A method of claim 16, characterized in that, at the step (b) of identifying the vesicle-associated proteins, the method includes:
(i) analysing tissue-resident extracellular vesicles and lipoproteins;
(ii) processing the isolated tissue-resident extracellular vesicles for isolating digested vesicle-associated peptides therefrom;
(iii) separating and analysing the digested vesicle-associated peptides; and
(iv) quantifying vesicle-associated proteins corresponding to the vesicle-associated peptides.

23. A method of claim 16, characterized in that, at the step (e) of comparing the vesicle-associated protein profiles, the method includes:
(a) obtaining the created vesicle-associated protein profiles for the identified vesicle-associated proteins in the tissue sample of the subject;
(b) obtaining the pre-determined vesicle-associated protein profiles of the tumour tissue samples and/or non-tumour tissue samples associated with the presence or absence of colon cancer, or risk of developing colon cancer; and
(c) checking if the created vesicle-associated protein profiles for the identified vesicle-associated proteins in the tissue sample of the subject matches with the pre-determined vesicle-associated protein profiles of the tumour tissue samples and/or non-tumour tissue samples.

24. A method of claim 16, characterized in that, at the step (e) of comparing the vesicle-associated protein profiles, the method includes employing at least one of a nanoFCM analysis, ELISA, alphaLISA, FACS, fluorescent correlation microscopy and immune-electron microscopy.

25. A method of claim 24, characterized in that the quantified vesicle-associated proteins is selected from a group consisting of: FAP, MGAT5, ST6GAL1, SCD, CHST14, DPEP1, NUP210, NFXL1, CHPF2, CHSY1, FUT6, CERS6, GALNT6, MMP14, AGRN, ICAM1, SEL1L3, FAT1, FCGR1A, FKBP11, DDX46, GBP1, TMCO1, EPHB3, MME, NDC1, TMEM2, LILRB3, CYP4F3, STT3B, DNAJA1, TMEM214, TMEM63A, FUT8, TAPBP, EXT2, CERS2, GGCX, CEACAM5, RPL15, FUT4, GLCE, MAN2A2, RHBDF2, TMX2, ENTPD6, FAM57A, CHMP3, HM13, GPAA1, SEC62, and IKBIP.

26. A method of claim 16, characterized in that the vesicle-associated proteins are selected from a group consisting of: CD63, CD81, Flotillin-1, TSG101, FN1, collagen alpha-1 (XIII) chain (COL12Aa), prolyl endopeptidase fibroblast activating factor (FAP), DEFA1, PADI4, CHPF2, CHST14, GPA33, MMP14, TMEM2, CD9, Alix, TSG101, Annexin A5, CHMP1A, ICAM1, EPHB3-1, EPHB3-2, TMEM2-1, TMEM2-2,CHMP1B, CHMP2A, CHMP2B, CHMP3, CHMP4A, CHMP4A, CHMP5, CHMP6, RAB2A, RAB2B, RAB5A, RAB5B, RAB5C, RAB7A, RAB11B, RAB27A, RAB27B and RAB35.

27. A system for identifying colon cancer-specific vesicle-associated proteins from a tissue sample of a subject, characterized in that the system includes:
(a) a kit for isolating tissue-resident extracellular vesicles from the tissue sample;
(b) a mass spectrometer for identifying vesicle-associated proteins associated with the isolated tissue-resident extracellular vesicles;
(c) a database having stored therein pre-determined vesicle-associated protein profiles of the tumour tissue samples and/or non-tumour tissue samples; and
(d) a computing unit in communication with the database, the computing unit including memory stored with executable codes operable to:
(i) quantify the identified vesicle-associated proteins to identify one or more major vesicle-associated proteins,
(ii) create vesicle-associated protein profiles for the identified vesicle-associated proteins, and
(iii) compare the vesicle-associated protein profiles for the identified vesicle-associated proteins with the pre-determined vesicle-associated protein profiles of the tumour tissue samples and/or non-tumour tissue samples.

28. A system of claim 27, characterized in that the computing unit is further operable to obtain intensity information of the identified vesicle-associated proteins for quantification, by employing a labelling tool.

29. A system of claim 27, characterized in that the kit comprises:
(a) a laboratory equipment for isolating the tissue-resident extracellular vesicles from a tissue sample, wherein the laboratory equipment comprises any of: a surgical arrangement, an RT-PCR, test tubes, pipettes, assay plates, a centrifuge, a 70-micrometer filter, a density gradient, a quantification system, an electron microscope, an analyser, a mass spectrometer;
(b) one or more reagents selected from a group consisting of: RPMI medium, DNase 1°0 , Collagenase D®, PBS medium, OptiPrep™ SDS, digestion buffer (trypsin/sodium deoxycholate), dithiothreitol, urea, TMT 11-plex isobaric mass tagging reagents®, TFA, ammonium formate buffer (formic acid), acetonitrile; (c) an epitope-specific binder against colon cancer-associated vesicle-associated proteins; and
(d) at least one colon cancer-associated marker detection agent.

30. A computer program product comprising non-transitory computer-readable storage medium having computer-readable instructions stored thereon, the computer-readable instructions being executable by a computerized device comprising processing hardware to execute a method of claim 16.
